(19) United States
(12) Patent Application Publication
(10) Pub. No.: US 2023/0407329 A1
(43) Pub. Date: Dec. 21, 2023

(54) IMPROVED AAV PRODUCTION USING POTASSIUM CHLORIDE

(71) Applicant: University of Florida Research Foundation, Incorporated, Gainesville, FL (US)

(72) Inventors: Nathalie Clement, Gainesville, FL (US); Laura Adamson, Gainesville, FL (US)

(73) Assignee: University of Florida Research Foundation, Incorporated, Gainesville, FL (US)

(21) Appl. No.: 18/037,893

(22) PCT Filed: Nov. 19, 2021
(86) PCT No.: PCT/US2021/060046

§ 371 Date: May 19, 2023

Related U.S. Application Data
(60) Provisional application No. 63/116,770, filed on Nov. 20, 2020.

(51) Int. Cl. C12N 15/86

(52) U.S. Cl. CPC C12N 15/86; C12N 2710/16644; C12N 2750/14143; C12N 2750/14151; C12N 2500/12

(57) ABSTRACT

Provided herein are methods of improving rAAV production in mammalian cells via the manipulation of a concentration of potassium chloride present in the media in which the mammalian cells are cultured and/or in which the rAAV particles are produced. The concentration of potassium chloride present in such media may be manipulated by, for example, supplementing a base medium with additional potassium chloride either before, at the same time as, or after the cells are contacted (e.g., infected) with one or more viral vectors (e.g., recombinant Herpes Simplex Virus (rHSV) vectors). Some embodiments of the present invention further contemplate supplementing the base medium with additional potassium chloride and sodium chloride in combination.

### CROSS REFERENCE TO RELATED APPLICATIONS

This application claims priority to U.S. provisional Application No. 63/116,770, filed Nov. 20, 2020, the content of which is herein incorporated by reference in its entirety.

### SEQUENCE LISTING

In accordance with 37 C.F.R. 1.52(e)(5), the present specification makes reference to a Sequence Listing (submitted electronically as a .txt file named “U120270060WO00-SEQ”). The .txt file was generated on Nov. 17, 2021, and is 707 bytes in size. The Sequence Listing is herein incorporated by reference in its entirety.

### BACKGROUND

Recombinant adeno-associated viral vectors (rAAV) have become a powerful research and clinical tool due to their ability to provide in vivo long-term gene expression. As the uses for rAAV grow, so does the need for large-scale manufacturing methods capable of generating high titers of high-quality vector. Several AAV vectors are currently in clinical development, and at least three AAV vectors are now commercially available. Yet the widespread full-scale clinical use of rAAV, in view of commercialization, has long been hampered by manufacturing limitations that have appeared as one of the greatest challenges undermining the production of large quantities of rAAV needed for clinical trials. Therefore, there is a need for techniques to improve the productivity and yield of rAAV vector particles produced at a large scale.

### SUMMARY

Provided herein are methods of improving rAAV production in mammalian cells via the manipulation of a concentration of potassium chloride present in the media in which the mammalian cells are cultured and/or in which the rAAV particles are produced. The concentration of potassium chloride present in such media may be manipulated by, for example, supplementing a base medium with additional potassium chloride either before, at the same time as, or after the cells are contacted (e.g., infected) with one or more viral vectors (e.g., recombinant Herpes Simplex Virus (rHSV) vectors that are used for rAAV production).

The art has long recognized that, while certain concentrations of salt added to cell culture media improve cell growth, an inhibitory effect forms as salt concentrations increase. See, e.g., H. Eagle (1956), The Salt Requirement of Mammalian Cells in Tissue Culture, Arc Biochem and Biophys, 61: 356-66. Surprisingly, the present inventors discovered a large improvement in rAAV production when the infection and/or production medium was supplemented with potassium chloride (KCl). In some embodiments, the total concentrations of KCl (e.g., the KCl present in the base medium+the supplemented KCl) at which such improvements were observed meet or even exceed those concentrations previously reported to be inhibitory.

Accordingly, aspects of the present disclosure include a method of producing rAAV in mammalian cells. In some embodiments, the method comprises: (i) culturing mammalian cells in a medium; (ii) adding one or more viral vectors encoding recombinant AAV (rAAV) genes and/or genes of interest into the medium; and (iii) supplementing the medium with at least 5 mM potassium chloride.

In some embodiments, the mammalian cells are HEK293 cells, BHK cells, or HeLa cells. In some embodiments, the recombinant AAV genes comprise one or more of AAV rep genes and/or AAV cap genes.

In some embodiments, the medium is supplemented with between 5 and 100 mM potassium chloride. In some embodiments, the medium is supplemented with between 20 and 100 mM potassium chloride. In some embodiments, the medium is supplemented with between and 100 mM potassium chloride. In some embodiments, the medium is supplemented with between 30 and 50 mM potassium chloride. In some embodiments, the medium is supplemented with 40 mM potassium chloride.

In some embodiments, the methods of the present disclosure further comprise: (iv) supplementing the medium with 60 mM of sodium chloride.

In some embodiments, the adding of step (ii) and supplementing of steps (iii) and/or (iv) are performed at the same time. In some embodiments, the supplementing of steps (iii) and/or (iv) is performed before the step of adding. In some embodiments, the supplementing of steps (iii) and/or (iv) is performed 2 hours before the step of adding. In some embodiments, the supplementing of steps (iii) and/or (iv) is performed after the step of adding. In some embodiments, the supplementing of steps (iii) and/or (iv) is performed between 1 minute and 8 hours after the step of adding. In some embodiments, the supplemented potassium chloride and/or sodium chloride remains present for between 8 and 14 hours after the time at which the mammalian cells are contacted with the one or more viral vectors.

In some embodiments, the methods of the present disclosure further comprise a step of diluting or changing the medium, such that the concentration of potassium chloride is decreased. In some embodiments, the methods of the present disclosure further comprise a step of diluting or changing the medium such that the concentration of sodium chloride is decreased. In some embodiments, the diluting or changing the medium occurs between 0.5 and 8 hours after the time at which the mammalian cells are contacted with the one or more viral vectors.

In some embodiments, the one or more viral vectors are one or more rHSV vectors. In some embodiments, the one or more rHSV vectors comprise a first rHSV encoding a gene of interest flanked by AAV ITRs, and a second rHSV encoding AAV rep and cap genes. In some embodiments, the first and second rHSVs are contacted to the cells together (e.g., simultaneously). In some embodiments, the AAV ITRs are AAV-2 ITRs. In some embodiments, the AAV ITRs are from AAV-1, AAV-3, AAV-4, AAV-5, AAV-6, AAV-7, AAV-8, AAV-9, AAV-10, AAV-11, AAV-12, or any other AAV serotype. In some embodiments, the AAV cap gene is an AAV-1, AAV-3, AAV-4, AAV-5, AAV-6, AAV-7, AAV-8, AAV-9, AAV-10, AAV-11, AAV-12, or any other AAV serotype cap gene. In some embodiments, the AAV rep and AAV cap genes are operably linked to a promoter.

As described herein, inventors of this disclosure have discovered that salt (e.g., KCl) is a critical component of an inoculum that improves yields in the production of rAAV in suspension-adapted cells, but that is not adjusted routinely. However, it should be appreciated that methods provided herein can be advantageous in the production of rAAV in adherent cells as well. Accordingly, in some embodiments, the mammalian cells are cultured in adherent format or suspension format. In some embodiments, the mammalian cells are cultured on micro-carriers that are in suspension.

Aspects of the present disclosure include a method of producing rAAV in mammalian cells. In some embodiments, the method comprises contacting mammalian cells with one or more viral vectors encoding recombinant AAV (rAAV) genes and/or genes of interest in the presence of at least 6 mM potassium chloride (e.g., including the supplemented potassium chloride and the potassium chloride which may or may not be already present in the base media).

In some embodiments, the potassium chloride is present in the medium in which the cells are contacted with the one or more viral vectors to introduce recombinant AAV genes (the infection medium) and/or genes of interest. In some embodiments, the potassium chloride is present in the medium in which the cells produce rAAV particles (the producer medium). In some embodiments, the producer medium is the same as the infection medium.

In some embodiments, the recombinant AAV genes comprise one or more of AAV rep genes and/or AAV cap genes.

In some embodiments, the mammalian cells are contacted in the presence of between 6 and 100 mM potassium chloride (e.g., including the supplemented potassium chloride and the potassium chloride which may or may not be already present in the base media). In some embodiments, the mammalian cells are contacted in the presence of between 20 and 100 mM potassium chloride. In some embodiments, the mammalian cells are contacted in the presence of between 30 and 100 mM potassium chloride. In some embodiments, the mammalian cells are contacted in the presence of between 30 and 50 mM potassium chloride. In some embodiments, the mammalian cells are contacted in the presence of 45 mM potassium chloride.

In some embodiments, the methods of the present invention further comprise a step of contacting the mammalian cells with 100-200 mM (e.g., 170 mM) sodium chloride.

In some embodiments, the potassium chloride and/or sodium chloride is present before, at, or after the time at which the mammalian cells are contacted with the one or more viral vectors. In some embodiments, the potassium chloride and/or sodium chloride is present 2 hours before the time at which the mammalian cells are contacted with the one or more viral vectors. In some embodiments, the potassium chloride and/or sodium chloride is present at the time at which the mammalian cells are contacted with the one or more viral vectors. In some embodiments, the potassium chloride and/or sodium chloride is present between 1 minute and 8 hours after the time at which the mammalian cells are contacted with the one or more viral vectors. In some embodiments, the potassium chloride and/or sodium chloride remains present for between 8 and 14 hours after the time at which the mammalian cells are contacted with the one or more viral vectors.

In some embodiments, the methods of the present invention further comprise a step of diluting or changing the medium such that the concentration of potassium chloride is decreased. In some embodiments, the methods of the present invention further comprise a step of diluting or changing the medium such that the concentration of sodium chloride is decreased. In some embodiments, the diluting or changing the medium occurs between 0.5 and 8 hours after the time at which the mammalian cells are contacted with the one or more viral vectors.

In some embodiments, the one or more viral vectors are one or more rHSV vectors. In some embodiments, the one or more rHSV vectors comprise a first rHSV encoding a gene of interest flanked by AAV ITRs, and a second rHSV encoding AAV rep and cap genes. In some embodiments, the first and second rHSVs are contacted to the cells together (e.g., simultaneously). In some embodiments, the AAV ITRs are AAV-2 ITRs. In some embodiments, the AAV ITRs are from AAV-1, AAV-3, AAV-4, AAV-5, AAV-6, AAV-7, AAV-8, AAV-9, AAV-10, AAV-11, AAV-12, or any other AAV serotype. In some embodiments, the AAV cap gene is an AAV-1, AAV-3, AAV-4, AAV-5, AAV-6, AAV-7, AAV-8, AAV-9, AAV-10, AAV-11, AAV-12, or any other AAV serotype cap gene. In some embodiments, the AAV rep and AAV cap genes are operably linked to a promoter.

In some embodiments, the mammalian cells are cultured in adherent format or suspension format. In some embodiments, the mammalian cells are cultured on micro-carriers that are in suspension. In some embodiments, the mammalian cells are HEK293 cells, BHK cells, or HeLa cells.

Aspects of the present disclosure include a method of producing rAAV in mammalian cells. In some embodiments, the method comprises: (i) supplementing a medium with at least 5 mM potassium chloride; and (ii) contacting the mammalian cells with one or more viral vectors encoding recombinant AAV (rAAV) genes and/or genes of interest in the supplemented medium.

In some embodiments, the medium is the medium in which the cells are contacted with the one or more viral vectors to introduce recombinant AAV genes (the infection medium) and/or genes of interest. In some embodiments, the medium is the medium in which the cells produce rAAV particles (the producer medium). In some embodiments, the producer medium is the same as the infection medium.

In some embodiments, the recombinant AAV genes comprise one or more of AAV rep genes and/or AAV cap genes. In some embodiments, the medium is supplemented with between 5 and 100 mM potassium chloride. In some embodiments, the medium is supplemented with between 20 and 100 mM potassium chloride. In some embodiments, the medium is supplemented with between 30 and 100 mM potassium chloride. In some embodiments, the medium is supplemented with between 30 and 50 mM potassium chloride. In some embodiments, the medium is supplemented with 40 mM potassium chloride.

In some embodiments, the methods of the present disclosure further comprise a step of supplementing the medium with 60 mM sodium chloride. In some embodiments, the medium is supplemented before, at, or after the time at which the mammalian cells are contacted with the one or more viral vectors. In some embodiments, the medium is supplemented 2 hours before the time at which the mammalian cells are contacted with the one or more viral vectors. In some embodiments, the medium is supplemented at the time at which the mammalian cells are contacted with the one or more viral vectors. In some embodiments, the medium is supplemented between 1 minute and 8 hours after the time at which the mammalian cells are contacted with the one or more viral vectors. In some embodiments, the supplemented potassium chloride and/or sodium chloride remains present for between 8 and 14 hours after the time at which the mammalian cells are contacted with the one or more viral vectors.

In some embodiments, the methods of the present disclosure further comprise a step of diluting or changing the medium such that the concentration of potassium chloride is decreased. In some embodiments, the methods of the present disclosure further comprise a step of diluting or changing the medium such that the concentration of sodium chloride is decreased. In some embodiments, the diluting or changing the medium occurs between 0.5 and 8 hours after the time at which the mammalian cells are contacted with the one or more viral vectors.

In some embodiments, the one or more viral vectors are one or more rHSV vectors. In some embodiments, the one or more rHSV vectors comprise a first rHSV encoding a gene of interest flanked by AAV ITRs, and a second rHSV encoding AAV rep and cap genes. In some embodiments, the first and second rHSVs are contacted to the cells together (e.g., simultaneously). In some embodiments, the AAV ITRs are AAV-2 ITRs. In some embodiments, the AAV ITRs are from AAV-1, AAV-3, AAV-4, AAV-5, AAV-6, AAV-7, AAV-8, AAV-9, AAV-10, AAV-11, AAV-12, or any other AAV serotype. In some embodiments, the AAV cap gene is an AAV-1, AAV-3, AAV-4, AAV-5, AAV-6, AAV-7, AAV-8, AAV-9, AAV-10, AAV-11, AAV-12, or any other AAV serotype cap gene. In some embodiments, the AAV rep and AAV cap genes are operably linked to a promoter.

In some embodiments, the mammalian cells are cultured in adherent format or suspension format. In some embodiments, the mammalian cells are cultured on micro-carriers that are in suspension. In some embodiments, the mammalian cells are HEK293 cells, BHK cells, or HeLa cells.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. It is to be understood that the data illustrated in the drawings in no way limit the scope of the disclosure.

FIG. 1 shows one embodiment of how rAAV particles are produced as disclosed herein.

FIG. 2 shows production of rAAV particles comprising GFP in different cell types.

FIGS. 3A to 3C show the scalability of rAAV production in EXPI293F™ cells.

FIG. 4 shows the theoretical scalability of the HSV system.

FIG. 5 shows that salt supplementation can occur 6 hours (gray-lined bars) after HSV co-infection in both adherent format (HEK293) or suspension format (EXPI293F™) without affecting the overall yield (black bars indicate that salt was added at the time of HSV co-infection) as measured by the total vector genomes.

FIG. 6 shows the effect of timing on HSV co-infection.

FIGS. 7A to 7D show the effect of potassium chloride (KCl) on AAV titers in comparison to sodium chloride (NaCl).

FIGS. 8A and 8B show the effect of salt combination on AAV titers.

FIGS. 9A to 9C show the effect of NaCl and KCl on cell growth, size and viability during AAV production.

FIG. 10 shows the timing of salt incubation for AAV titer increase.

FIGS. 11A to 11E demonstrate the effect of osmolality on AAV titers.

FIG. 12 shows the effect of salt on AAV yields when produced by transfection.

FIG. 13 shows the effect of supplementation prior to infection. The average of two independent experiments is shown.

FIG. 14 shows the effect of potassium phosphates K₂HPO₄ and KH₂PO₄ on AAV yield (vector genomes; vg), as compared to no supplement, NaCl (60 mM), and KCl (40 mM).

FIG. 15 shows the effect of sodium phosphate Na₂HPO₄ on AAV yield (vector genomes; vg), as compared to no supplement, NaCl (60 mM), and KCl (40 mM).

FIG. 16 shows the effect of K₂HPO₄, KH₂PO₄, Na₂HPO₄ on AAV production (infectious unit titers), as compared to no supplement, NaCl (60 mM), and KCl (40 mM).

FIG. 17 shows the effect of potassium phosphates K₂HPO₄ and KH₂PO₄ on AAV yield (vector genomes; vg) for supplemented concentrations ranging from 1 to 10 mM, as compared to no supplement, NaCl (60 mM), and KCl (40 mM).

FIG. 18 shows the effect of sodium phosphate Na₂HPO₄ on AAV yield (vector genomes; vg) for supplemented concentrations ranging from 1 to 10 mM, as compared to no supplement, NaCl (60 mM), and KCl (40 mM).

### DETAILED DESCRIPTION

As the effectiveness of rAAV-based therapies becomes more evident by clinical outcomes in a number of indications, so does the demand for scalable, more efficient, and high-yield methods of rAAV particle production.

Usually, rAAV production involves (1) culturing cells, (2) introducing AAV genes and any genes desired to be packaged to the cells, and (3) allowing the cells to produce or package rAAV. The last step is followed by harvesting rAAV particles and subsequent purification steps. AAV genes and any genes desired to be packaged into rAAV particles may be introduced to cells by either transfection methods (e.g., using plasmid vectors and a transfection agent) or infection methods (e.g., using one or more viral vectors, such as HSV vectors, that encode one or more AAV genes and/or genes of interest to be packaged). If an infection method is utilized, as in the present disclosure, the cells are contacted with one or more viral vectors which infect the cells to deliver AAV genes and any genes desired to be packaged. Cells are said to be “infected” at the time when the cells are first contacted with (e.g., introduced to) the infection reagents (e.g., viral vectors).

The media used in the three stages of rAAV production may be the same or different in one or more of the stages of production. Herein, media used in these stages of rAAV production may be referred to as “culture medium”, “infection medium”, and “producer medium”. It will be understood that, unless otherwise specified, “medium” or “media” may refer to any or all of culture, infection, and/or producer media. Accordingly, in some embodiments, the methods of the present invention comprise (1) culturing mammalian cells in a first medium (e.g., a culture medium), (2) contacting the cells with a second medium, wherein the second medium comprises one or more viral vectors (e.g., an infection medium), and (3) producing rAAV particles in a third medium (e.g., a production medium). In some embodiments, cells are first contacted with the infection reagents upon being contacted with the infection medium.

Provided herein are methods of improving rAAV production in mammalian cells via the manipulation of a concentration of potassium chloride present in the media in which the mammalian cells are cultured and/or in which the rAAV particles are produced. The concentration of potassium chloride present in such media may be manipulated by, for example, supplementing a base medium with additional potassium chloride either before, at the same time as, or after the cells are contacted (e.g., infected) with one or more viral vectors (e.g., recombinant Herpes Simplex Virus (rHSV) vectors). Some embodiments of the present invention further contemplate supplementing the base medium with additional potassium chloride and sodium chloride in combination.

Improving rAAV Production by Increasing Potassium Chloride Concentration in Media

Provided herein are methods of producing rAAV in mammalian cells by contacting the mammalian cells with one or more viral vectors encoding AAV rep and cap genes in the presence of potassium chloride (KCl). In some embodiments, the KCl is present in the medium in which the cells are contacted (e.g., infected) to introduce AAV rep and cap genes and/or genes to be packaged into rAAV particles (e.g., the infection medium). In some embodiments, the KCl is present in the medium in which cells produce rAAV particles (e.g., the producer medium). In some embodiments, the producer medium is the same as the infection medium.

Accordingly, in some embodiments, the methods of the present invention comprise (1) culturing mammalian cells in a first medium (e.g., a culture medium), (2) contacting the cells with a second medium, wherein the second medium comprises one or more viral vectors (e.g., an infection medium), and (3) producing rAAV particles in a third medium (e.g., a production medium). In some embodiments, cells are first contacted with the infection reagents upon being contacted with the infection medium. It will be understood that the first, second, and third mediums may in some embodiments be characterized separately (e.g., as three separate media) due to each medium containing different components (e.g., KCl) or concentrations of components. For example, if KCl is added to the first medium, and the concentration of KCl in the medium is therefore increased, the resultant medium with the increased concentration of KCl would be considered a second medium.

In some embodiments, the KCl is present in the medium due to supplementation of KCl to the base medium. As used herein, a “base medium” is an un-supplemented medium in which cells are cultured, contacted with one or more viral vectors (e.g., infected), and/or in which rAAV particles are produced (e.g., a first medium). A base medium may in some embodiments be a commercially available medium, such as, for example, EXPI, DMEM, or other medium. Thus, in some embodiments, the base medium (e.g., the culture, infection, and/or producer media) is supplemented with KCl (e.g., creating a second medium). In some embodiments, the concentration of KCl in the medium is increased by supplementing the base medium with KCl. In some embodiments, KCl is supplemented to the medium by adding KCl in solid or crystalline form directly to the base medium. In some embodiments, a solution of KCl is added to base medium to supplement it.

Some aspects of the present invention therefore contemplate methods of producing rAAV in mammalian cells comprising supplementing a medium with potassium chloride, and contacting the mammalian cells with one or more viral vectors encoding recombinant AAV (rAAV) genes and/or genes of interest in the supplemented medium. Still other aspects include methods of producing rAAV in mammalian cells comprising culturing mammalian cells in a medium, adding one or more viral vectors encoding recombinant AAV (rAAV) genes and/or genes of interest into the medium, and supplementing the medium with potassium chloride.

In some embodiments, a concentration of KCl is increased (e.g., by supplementing) in the infection medium alone. In some embodiments, a concentration of KCl is increased (e.g., by supplementing) in the producer medium alone (e.g., after infection of the cells). In some embodiments, the concentration of KCl is increased in both infection and producer media. In some embodiments, KCl is present before, at, or after the time at which the mammalian cells are contacted (e.g., infected) with the one or more viral vectors. In some embodiments, concentration of KCl is increased (e.g., by supplementation) before, at, or after the time at which the mammalian cells are infected with the one or more viral vectors. The producer medium may then be added to the infection medium. In some embodiments, the KCl concentration of the producer medium that is added to the infection medium is increased either prior to, or after, addition to the infection medium. In some embodiments, the infection medium is exchanged for the producer medium (e.g., by centrifuging the cells). If the KCl concentration in the producer medium that replaces the infection medium is increased, it may be increased either prior to, at, or after the time the infection medium is replaced with the producer medium.

In some embodiments, two or more of the culture medium, infection medium, and producer medium are the same. For example, the culture medium may be the same as the infection medium. Vectors (e.g., viral vectors) used for infection may be introduced to cells without exchanging the culture medium for the infection medium, or without adding the infection medium. In some embodiments, the infection medium is the same as the producer medium. For example, no exchange or addition of media occurs after cells are contacted with one or more viral vectors (e.g., infected). In some embodiments, all three of culture, infection, and producer media are the same.

It is to be understood that by stating that more than one media are the “same”, it is meant that, absent supplementation according to the methods of the present invention, each of all the components of each medium in question are the same and in equal proportions or quantities, relative to the other medium(s). A non-limiting list of possible media components includes buffering agents (e.g., phosphates or acetates), nutrients (e.g., proteins, peptides or amino acids), carbohydrates, essential metals and minerals, indicators for pH change, antimicrobial agents, and gelling agents (e.g., agar).

It should be appreciated that salt may be a component of a base medium (e.g., commercially-available medium) prior to KCl supplementation. Further, the identity of the particular salt(s) and/or the salt concentration may be proprietary information belonging to a manufacturer and may thus be unknown to a user.

In some embodiments, a commercially available medium is adapted for cell growth. Increasing the salt (e.g., by adding KCl) during the infection and/or production stages can be beneficial. For example, the reported KCl concentration in DMEM of a commercial source may be 400 mg/L (5.37 mM), but an increase in rAAV vector production may be observed when 40 mM of KCl is added. This would correspond to a final concentration of approximately 45.37 mM KCl. However, other commercial media (e.g., EXPI media) contain unpublished and therefore unknown (e.g., unknown to the user) concentrations of all components in the media, including KCl. Through the inventors' work, it is found that a benefit in rAAV yield can be attained by KCl supplementation even for base media for which the salt concentration is unknown.

As used herein, “rAAV vectors” are used synonymously with “rAAV particles” or may also mean nucleic acid vectors that are used to produce rAAV particles.

### Yield Improvement

rAAV yields may be improved by using any one of the methods described herein compared to rAAV production processes that are otherwise the same, with the exception of a change in KCl concentration (e.g., via KCl supplementation). In some embodiments, the yield of rAAV production which includes KCl concentration changes may increase by 1.2 to 25 times (e.g., 1.2-19, 2-15, 3-12, 5-10, 6-8, or 19-25 times) compared to rAAV production processes wherein KCl concentration is not changed. In some embodiments, the yield of rAAV production which includes KCl concentration changes may increase by 2, 3, 4, 5, 6, 7, 8, 9, 10, 11, 12, 13, 14, 15, 16, 17, 18, 19, 20, 21, 22, 23, 24 or 25 times compared to rAAV production processes wherein KCl concentration is not changed.

In some embodiments, the yield of rAAV production which includes KCl concentration is increased one-fold, two-fold, three-fold, four-fold, five-fold, six-fold, seven fold, eight-fold, nine-fold, ten-fold, eleven-fold, twelve-fold, thirteen-fold, fourteen-fold, fifteen-fold, sixteen-fold, seventeen-fold, eighteen-fold, nineteen-fold, or twenty-fold compared to rAAV production processes wherein KCl concentration is not changed. In some embodiments, the yield of rAAV production which includes KCl concentration is increased nineteen-fold compared to rAAV production processes wherein KCl concentration is not changed.

In some embodiments, the yield of rAAV production which includes KCl concentration is increased by about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 101%, 102%, 103%, 104%, 105%, 106%, 107%, 108%, 109%, 110%, 111%, 112%, 113%, 114%, 115%, 116%, 117%, 118%, 119%, 120%, 121%, 122%, 123%, 124%, 125%, 126%, 127%, 128%, 129%, 130%, 131%, 132%, 133%, 134%, 135%, 136%, 137%, 138%, 139%, 140%, 141%, 142%, 143%, 144%, 145%, 146%, 147%, 148%, 149%, 150%, 151%, 152%, 153%, 154%, 155%, 156%, 157%, 158%, 159%, 160%, 161%, 162%, 163%, 164%, 165%, 166%, 167%, 168%, 169%, 170%, 171%, 172%, 173%, 174%, 175%, 176%, 178%, 179%, 180%, 181%, 182%, 183%, 184%, 185%, 186%, 187%, 188%, 189%, 190%, 191%, 192%, 193%, 194%, 195%, 196%, 197%, 198%, 199%, 200%, 250%, 300%, 400%, 500%, 1,000%, 1,500%, 2,000%, etc. compared to rAAV production processes wherein KCl concentration is not changed.

### Salt

The present invention is based on methods of contacting mammalian cells with one or more viral vectors encoding AAV rep and cap genes in the presence of KCl. The KCl may in some embodiments be already present in the medium. In some embodiments, the KCl is supplemented. In some embodiments, the KCl is present in the medium due to supplementation of KCl to the medium. In some embodiments, the supplementation of KCl to the medium results in an increase in KCl concentration in the medium. KCl may be present in or supplemented to any or all of the culture, infection, and/or producer media, as described herein, and may be present at varying concentrations.

Some aspects of the present invention contemplate methods of producing rAAV in mammalian cells comprising supplementing a medium with potassium chloride, and contacting the mammalian cells with one or more viral vectors encoding recombinant AAV (rAAV) genes and/or genes of interest in the supplemented medium. Still other aspects include methods of producing rAAV in mammalian cells comprising culturing mammalian cells in a medium, adding one or more viral vectors encoding recombinant AAV (rAAV) genes and/or genes of interest into the medium, and supplementing the medium with potassium chloride.

In some embodiments, the mammalian cells are contacted in the presence of at least 6 mM KCl (e.g., including the supplemented potassium chloride and the potassium chloride which may or may not be already present in the base media). In some embodiments, the mammalian cells are contacted in the presence of between 6 and 100 mM KCl. In some embodiments, the mammalian cells are contacted in the presence of between 20 and 100 mM KCl. In some embodiments, the mammalian cells are contacted in the presence of between 30 and 100 mM KCl. In some embodiments, the mammalian cells are contacted in the presence of between 30 and 50 mM KCl. In some embodiments, the mammalian cells are contacted in the presence of 45 mM KCl.

In some embodiments, a known volume of a known concentration of supplemented KCl (e.g., that which is added to a base medium) solution is added to a medium (e.g., a base medium, which may be an infection and/or producer medium) to result in a known concentration of supplemented KCl present in the medium. In some embodiments, “supplementing” a medium with a concentration (e.g., at least 5 mM) of KCl is accomplished by adding a known amount (e.g., a known volume of a known concentration) of KCl that results in an increase of the concentration of KCl present in the medium (e.g., the concentration of KCl in the medium will be raised by 5 mM, relative to the concentration of KCl in the medium prior to supplementation). In some embodiments, supplementing a medium with a concentration (e.g., at least 5 mM) of KCl is accomplished by adding a known amount (e.g., a known volume of a known concentration) of KCl that results in an increase of the concentration of KCl present in the medium (e.g., the concentration of KCl in the medium will be raised by 5 mM, relative to the concentration of KCl in the medium prior to supplementation), and which results in the total concentration of KCl in the medium being equal to the supplemented amount (e.g., the total concentration of KCl in the medium will be 5 mM).

In some embodiments, the concentration of supplemented KCl present in the medium is at least 5 mM. In some embodiments, the concentration of supplemented KCl present in the medium is between 5 mM and 100 mM. In some embodiments, the concentration of supplemented KCl present in the medium is between 20 mM and 100 mM. In some embodiments, the concentration of supplemented KCl present in the medium is between 30 mM and 100 mM. In some embodiments, the concentration of supplemented KCl present in the medium is between 5 mM and 20 mM. In some embodiments, the concentration of supplemented KCl present in the medium is between 10 mM and 25 mM. In some embodiments, the concentration of supplemented KCl present in the medium is between 25 mM and 30 mM. In some embodiments, the concentration of supplemented KCl present in the medium is between 20 mM and 35 mM. In some embodiments, the concentration of supplemented KCl present in the medium is between 25 mM and 40 mM. In some embodiments, the concentration of supplemented KCl present in the medium is between 30 mM and 45 mM. In some embodiments, the concentration of supplemented KCl present in the medium is between 35 mM and 50 mM. In some embodiments, the concentration of supplemented KCl present in the medium is between 40 mM and 55 mM. In some embodiments, the concentration of supplemented KCl present in the medium is between 45 mM and 60 mM. In some embodiments, the concentration of supplemented KCl present in the medium is between 30 mM and 50 mM. In some embodiments, the concentration of supplemented KCl present in the medium is 40 mM. However, it should be appreciated that other concentrations of supplemented KCl may be used in one or more medium.

In some embodiments, the medium may already contain an initial salt concentration (e.g., of KCl and/or of a different salt) which may be known or unknown. In such instances, the concentration of the supplemented KCl present in the medium is an additional salt content of the medium relative to the initial salt content prior to the addition of the supplemented KCl. In some embodiments, the supplemented KCl is the only salt present in the medium and the concentration of supplemented KCl present in the medium is the actual salt concentration of the medium. In some embodiments, no KCl is present in the medium prior to supplementation.

In some embodiments, the concentration of KCl is supplemented such that the total concentration of KCl present in a supplemented medium is between 5 mM and 105 mM. In some embodiments, the concentration of KCl is supplemented such that the total concentration of KCl present in a supplemented medium is between 20 mM and 105 mM. In some embodiments, the concentration of KCl present in a supplemented medium is supplemented such that the total concentration of KCl is between 30 mM and 105 mM. In some embodiments, the concentration of KCl present in a supplemented medium is supplemented such that the total concentration of KCl is between 5 mM and 25 mM. In some embodiments, the concentration of KCl present in a supplemented medium is supplemented such that the total concentration of KCl is between 10 mM and 30 mM. In some embodiments, the concentration of KCl present in a supplemented medium is supplemented such that the total concentration of KCl is between 25 mM and 35 mM. In some embodiments, the concentration of KCl present in a supplemented medium is supplemented such that the total concentration of KCl is between 20 mM and 40 mM. In some embodiments, the concentration of KCl present in a supplemented medium is supplemented such that the total concentration of KCl is between 25 mM and 45 mM. In some embodiments, the concentration of KCl present in a supplemented medium is supplemented such that the total concentration of KCl is between 30 mM and 50 mM. In some embodiments, the concentration of KCl present in a supplemented medium is supplemented such that the total concentration of KCl is between 30 mM and 55 mM. In some embodiments, the concentration of KCl present in a supplemented medium is supplemented such that the total concentration of KCl is between 30 mM and 60 mM. In some embodiments, the concentration of KCl present in a supplemented medium is supplemented such that the total concentration of KCl is between 35 mM and 60 mM. In some embodiments, the concentration of KCl present in a supplemented medium is supplemented such that the total concentration of KCl is between 40 mM and 60 mM. In some embodiments, the concentration of KCl present in a supplemented medium is supplemented such that the total concentration of KCl is between 40 mM and 55 mM. In some embodiments, the concentration of KCl present in a supplemented medium is supplemented such that the total concentration of KCl is 45 mM.

It should therefore be appreciated that the total concentration of salt may be a combined concentration of the supplemented KCl and one or more additional salts (e.g., 2-5, 5-10, or more different salts), if such additional salts are present in the medium prior to KCl supplementation, or if such additional salts are also supplemented. In some embodiments, at least 109.5 mM of sodium chloride (NaCl) is present in the medium prior to KCl supplementation. In some embodiments, at least 110 mM of sodium chloride (NaCl) is present in the medium prior to KCl supplementation. Thus, in some embodiments, the method further comprises contacting the mammalian cells in the presence of at least 105 mM (e.g., at least 109.5 mM or at least 110 mM of NaCl). In some embodiments, less than 200 mM of sodium chloride (NaCl) is present in the medium prior to KCl supplementation. In some embodiments, less than 150 mM of sodium chloride (NaCl) is present in the medium prior to KCl supplementation. In some embodiments, less than 100 mM of sodium chloride (NaCl) is present in the medium prior to KCl supplementation. In some embodiments, less than 50 mM of sodium chloride (NaCl) is present in the medium prior to KCl supplementation. In some embodiments, no sodium chloride (NaCl) is present in the medium prior to KCl supplementation.

In some embodiments, the concentration of KCl is supplemented such that the total concentration of KCl and NaCl present in a supplemented medium is between 125 and 250 mM. In some embodiments, the concentration of KCl is supplemented such that the total concentration of KCl and NaCl present in a supplemented medium is between 130 and 200 mM. In some embodiments, the concentration of KCl present in a supplemented medium is supplemented such that the total concentration of KCl and NaCl is between 135 and 180 mM. In some embodiments, the concentration of KCl present in a supplemented medium is supplemented such that the total concentration of KCl and NaCl is between 200 and 250 mM. In some embodiments, the concentration of KCl present in a supplemented medium is supplemented such that the total concentration of KCl and NaCl is between 200 and 300 mM. In some embodiments, the concentration of KCl present in a supplemented medium is supplemented such that the total concentration of KCl and NaCl is between 300 and 400 mM. In some embodiments, the concentration of KCl present in a supplemented medium is supplemented such that the total concentration of KCl and NaCl is between 400 and 500 mM. In some embodiments, the concentration of KCl present in a supplemented medium is supplemented such that the total concentration of KCl and NaCl is between 500 mM and 1 M.

Some embodiments contemplate contacting the mammalian cells in the presence of a second supplemented salt, in addition to KCl. Thus, in some embodiments, a concentration of a second salt, in addition to KCl, is supplemented (e.g., increased) in a medium (e.g., a culture medium, infection medium, and/or producer medium). In some embodiments, the second supplemented salt is NaCl. In some embodiments, the mammalian cells are contacted in the presence of between 100 and 200 mM NaCl. In some embodiments, the mammalian cells are contacted in the presence of between 100 and 150 mM NaCl. In some embodiments, the mammalian cells are contacted in the presence of between 110 and 200 mM NaCl. In some embodiments, the mammalian cells are contacted in the presence of between 120 and 200 mM NaCl. In some embodiments, the mammalian cells are contacted in the presence of between 130 and 200 mM NaCl. In some embodiments, the mammalian cells are contacted in the presence of between 140 and 200 mM NaCl. In some embodiments, the mammalian cells are contacted in the presence of between 150 and 180 mM NaCl. In some embodiments, the mammalian cells are contacted in the presence of 170 mM NaCl.

In some embodiments, the medium is supplemented with at least 15 mM of sodium chloride. In some embodiments, the medium is supplemented with at least 16 mM of sodium chloride. In some embodiments, the medium is supplemented with between 16 and 100 mM sodium chloride. In some embodiments, the medium is supplemented with between 16 and 90 mM sodium chloride. In some embodiments, the medium is supplemented with between 20 and mM sodium chloride. In some embodiments, the medium is supplemented with between 30 and 60 mM sodium chloride. In some embodiments, the medium is supplemented with between and 60 mM sodium chloride. In some embodiments, the medium is supplemented with between 50 and 60 mM sodium chloride. In some embodiments, the medium is supplemented with 60 mM sodium chloride.

In some embodiments, the second supplemented salt is an inorganic salt. In some embodiments, the second supplemented salt is an alkali halide. Alkali halides are salts comprised of an alkali metal (e.g., lithium, sodium, potassium, rubidium, and caesium) and a halogen (e.g., fluorine, chlorine, bromine, and iodine). In some embodiments, an alkali halide is sodium chloride (NaCl). In some embodiments, an alkali halide is comprised of an alkali metal selected from Li, Na, K, Rb, and Cs, and a halogen selected from F, Cl, Br, and I. In some embodiments, a second supplemented salt is an alkaline earth halide. In some embodiments, an alkaline earth halide is comprised of an alkaline earth metal selected from Be, Mg, Ba, and Ca, and a halogen selected from F, Cl, Br, and I. In some embodiments, a second supplemented salt is a metal selenide, metal hydroxide, metal oxide, metal phosphate, metal silicate, metal borate, metal carbonate, metal nitrate, or a metal sulfate. In some embodiments, the second supplemented salt is sodium chloride (NaCl).

In some embodiments, the second supplemented salt is one selected from the group consisting of aluminum chloride, magnesium chloride, lithium selenide, sodium carbonate, lithium chloride, sodium hydrogen phosphate, sodium metasilicate, strontium hydroxide, trisodium phosphate, potassium fluoride, magnesium sulfate, calcium chloride, sodium sulfate, aluminum sulfate, sodium tetraborate, magnesium sulfate, magnesium bromide, rubidium aluminum sulfate, barium hydroxide, potassium aluminum sulfate, magnesium nitrate, sodium hydrogen phosphate, nickel sulfate, zinc sulfate, beryllium sulfate, lithium nitrate, strontium chloride, zinc nitrate, sodium pyrophosphate, calcium bromide, copper sulfate, copper nitrate, aluminum nitrate, sodium tetraborate, silver fluoride, calcium iodide, lithium bromide, lithium iodide, strontium bromide, calcium nitrate, strontium iodide, sodium bromide and strontium nitrate.

In some embodiments, a second supplemented salt is an organic salt. Some non-limiting examples of suitable organic second supplemented salts include sodium aluminum lactate, sodium acetate, sodium dehydroacetate, sodium butoxy ethoxy acetate, sodium caprylate, sodium citrate, sodium lactate, sodium dihydroxy glycinate, sodium gluconate, sodium glutamate, sodium hydroxymethane sulfonate, sodium oxalate, sodium phenate, sodium propionate, sodium saccharin, sodium salicylate, sodium sarcosinate, sodium toluene sulfonate, magnesium aspartate, calcium propionate, calcium saccharin, calcium d-saccharate, calcium thioglycolate, aluminum caprylate, aluminum citrate, aluminum diacetate, aluminum glycinate, aluminum lactate, aluminum methionate, aluminum phenosulfonate, potassium aspartate, potassium biphthalate, potassium bitartrate, potassium glycosulfate, potassium sorbate, potassium thioglycolate, potassium toluene sulfonate and magnesium lactate.

In some embodiments of any one of the methods described herein, the concentration of a second supplemented salt in an infection medium and/or producer medium is between 1-250 mM (e.g., 5-200 mM, 20-180 mM, 30-150 mM, 60-90 mM, 40-120 mM, 50-100 mM, or 70-80 mM). The “concentration of a second supplemented salt” is that which is added, and not the total concentration of a second supplemented salt (e.g., a salt other than KCl) in a medium.

In some embodiments, concentration of salt in an infection medium and/or producer medium is supplemented such that the total concentration of salt (e.g., KCl plus a second supplemented salt) is between 125 and 250 mM (e.g., 130-200 mM, 135-180 mM, 140-185 mM, or 145-180 mM).

In some embodiments, the salt concentration (e.g., KCl alone, or KCl plus a second supplemented salt) in the culture, infection, and producer media is increased such that the concentration of salt (e.g., KCl alone, or KCl plus a second supplemented salt) in the infection medium is higher than the concentration of salt (e.g., KCl alone, or KCl plus a second supplemented salt) in the culture medium. In some embodiments, the salt concentration in the culture, infection, and producer media is increased such that the concentration of salt (e.g., KCl alone, or KCl plus a second supplemented salt) in the infection medium is higher than the concentration of salt (e.g., KCl alone, or KCl plus a second supplemented salt) in the culture medium and the producer medium. In some embodiments, the concentration of salt (e.g., KCl alone, or KCl plus a second supplemented salt) in the infection medium and the producer medium is increased such that the concentration of salt (e.g., KCl alone, or KCl plus a second supplemented salt) in the infection medium and in the producer medium is higher than the concentration of salt (e.g., KCl alone, or KCl plus a second supplemented salt) in the culture medium.

In some embodiments, the concentration of a salt (e.g., potassium chloride) present in a medium (e.g., a base medium, or a supplemented medium) is measured using any method known in the art. Methods of measuring a salt concentration in a medium will be readily apparent to those of skill in the art.

### Timing

In some embodiments, KCl is present in the medium before the time at which the mammalian cells are contacted (e.g., infected) with the one or more viral vectors (e.g., infection of cells with AAV rep and cap genes, other helper genes, and/or one or more genes of interest). In some embodiments, KCl is present in the medium for at least 1 minute (e.g., 1-60, 5-45, or minutes, or 1-4, 2-3, or 1-2 hours, or up to 1 day) before the time at which the mammalian cells are contacted (e.g., infected) with the one or more viral vectors. In some embodiments, KCl is present 1 hour before the time at which the mammalian cells are contacted (e.g., infected) with the one or more viral vectors.

In some embodiments, KCl is present in the medium at the time at which the mammalian cells are contacted (e.g., infected) with the one or more viral vectors (e.g., infection of cells with AAV rep and cap genes, other helper genes, and/or one or more genes of interest).

In some embodiments, KCl is present in the medium after the time at which the mammalian cells are contacted (e.g., infected) with the one or more viral vectors (e.g., infection of cells with AAV rep and cap genes, other helper genes, and/or one or more genes of interest). In some embodiments, KCl is present in the medium for at least 1 minute (e.g., 1-60, 5-45, or minutes, or 1-10, 2-8, or 3-4 hours, or up to 1 day) after the time at which the mammalian cells are contacted (e.g., infected) with the one or more viral vectors. In some embodiments, KCl is present between 1 minute and 10 hours after the time at which the mammalian cells are contacted (e.g., infected) with the one or more viral vectors.

In some embodiments, KCl remains present in the medium after the time at which the mammalian cells are contacted (e.g., infected) with the one or more viral vectors (e.g., infection of cells with AAV rep and cap genes, other helper genes, and/or one or more genes of interest). In some embodiments, KCl remains present in the medium for at least 8 hours (e.g., 8-20, 10-18, or 14-16 hours, or 1-2 days) after the time at which the mammalian cells are contacted (e.g., infected) with the one or more viral vectors. In some embodiments, the KCl remains present for between 8 and 14 hours after the time at which the mammalian cells are contacted (e.g., infected) with the one or more viral vectors.

In some embodiments, the KCl is present in the medium due to supplementation of KCl to the medium. In some embodiments, the supplementation of KCl to the medium results in an increase in KCl concentration in the medium. In some embodiments, KCl concentration is increased (e.g., by supplementation) before the contacting (e.g., infection) of cells with AAV rep and cap genes, other helper genes, and/or one or more genes of interest. For example, if the culture medium is exchanged for the infection medium, KCl concentration may be increased in the infection medium (e.g., by supplementation) either immediately before contacting (e.g., infecting) the cells, at the time of contacting the cells, or immediately after contacting the cells.

In some embodiments, KCl concentration may be increased in the infection medium (e.g., by supplementing) a few hours (e.g., 1-8, 2-6, or 3-4 hours) or a few minutes (e.g., 1-60, 5-or 15-30 minutes) before the cells are contacted (e.g., infected) with one or more viral vectors. In some embodiments, KCl concentration is increased in the infection medium (e.g., by supplementing) a few hours (e.g., 1-8, 2-6, or 3-4 hours) or a few minutes (e.g., 1-60, 5-45, or minutes) after the cells are contacted (e.g., infected) with one or more viral vectors. In some embodiments, KCl concentration is increased in the culture medium (e.g., by supplementing) a few hours (e.g., 1-8, 2-6, or 3-4 hours) or a few minutes (e.g., 1-60, 5-45, or 15-30 minutes) before the cells are contacted (e.g., infected) with one or more viral vectors. In some embodiments, KCl concentration is increased (e.g., by supplementing) at the beginning of the rAAV production process, when the cells begin culturing or are in the growth phase. In some embodiments, KCl concentration is increased in the producer medium (e.g., by supplementing) a few hours (e.g., 1-8, 2-6, or 3-4 hours) or a few minutes (e.g., 1-60, 5-45, or 15-30 minutes) after the cells are contacted (e.g., infected) with one or more viral vectors.

In some embodiments, wherein NaCl is additionally present in and/or supplemented to the medium, NaCl is present in the medium before the time at which the mammalian cells are contacted (e.g., infected) with one or more viral vectors (e.g., infection of cells with AAV rep and cap genes, other helper genes, and/or one or more genes of interest). In some embodiments, NaC1 is present in the medium for at least 1 minute (e.g., 1-60, 5-45, or 15-30 minutes, or 1-4, 2-3, or 1-2 hours, or up to 1 day) before the time at which the mammalian cells are contacted (e.g., infected) with one or more viral vectors. In some embodiments, NaCl is present 1 hour before the time at which the mammalian cells are contacted (e.g., infected) with one or more viral vectors.

In some embodiments, NaCl is present in the medium at the time at which the mammalian cells are contacted (e.g., infected) with one or more viral vectors (e.g., infection of cells with AAV rep and cap genes, other helper genes, and/or one or more genes of interest).

In some embodiments, NaCl is present in the medium after the time at which the mammalian cells are contacted (e.g., infected) with one or more viral vectors (e.g., infection of cells with AAV rep and cap genes, other helper genes, and/or one or more genes of interest). In some embodiments, NaCl is present in the medium for at least 1 minute (e.g., 1-60, 5-45, or 15-minutes, or 1-10, 2-8, or 3-4 hours, or up to 1 day) after the time at which the mammalian cells are contacted (e.g., infected) with one or more viral vectors. In some embodiments, NaCl is present between 1 minute and 10 hours after the time at which the mammalian cells are contacted (e.g., infected) with one or more viral vectors.

In some embodiments, NaCl remains present in the medium after the time at which the mammalian cells are contacted (e.g., infected) with one or more viral vectors (e.g., infection of cells with AAV rep and cap genes, other helper genes, and/or one or more genes of interest). In some embodiments, NaCl remains present in the medium for at least 8 hours (e.g., 8-20, 10-18, or 14-16 hours, or 1-2 days) after the time at which the mammalian cells are contacted (e.g., infected) with one or more viral vectors. In some embodiments, the NaCl remains present for between 8 and 14 hours after the time at which the mammalian cells are contacted (e.g., infected) with one or more viral vectors.

In some embodiments, the NaCl is present in the medium due to supplementation of NaCl to the medium. In some embodiments, the supplementation of NaCl to the medium results in an increase in NaCl concentration in the medium. In some embodiments, NaCl concentration is increased (e.g., by supplementation) before the cells are contacted (e.g., infected) with AAV rep and cap genes, other helper genes, and/or one or more genes of interest. For example, if the culture medium is exchanged for the infection medium, NaCl concentration may be increased in the infection medium (e.g., by supplementation) either immediately before the cells are contacted, at the time the cells are contacted, or immediately after the cells are contacted.

In some embodiments, NaCl concentration may be increased in the infection medium (e.g., by supplementing) a few hours (e.g., 1-8, 2-6, or 3-4 hours) or a few minutes (e.g., 1-60, 5-45, or 15-30 minutes) before the cells are contacted (e.g., infected) with one or more viral vectors. In some embodiments, NaCl concentration is increased in the infection medium (e.g., by supplementing) a few hours (e.g., 1-8, 2-6, or 3-4 hours) or a few minutes (e.g., 1-60, 5-45, or minutes) after the cells are contacted (e.g., infected) with one or more viral vectors. In some embodiments, NaCl concentration is increased in the culture medium (e.g., by supplementing) a few hours (e.g., 1-8, 2-6, or 3-4 hours) or a few minutes (e.g., 1-60, 5-45, or minutes) before the cells are contacted (e.g., infected) with one or more viral vectors. In some embodiments, NaCl concentration is increased (e.g., by supplementing) at the beginning of the rAAV production process, when the cells begin culturing or are in the growth phase. In some embodiments, NaCl concentration is increased in the producer medium (e.g., by supplementing) a few hours (e.g., 1-8, 2-6, or 3-4 hours) or a few minutes (e.g., 1-60, 5-45, or minutes) after the cells are contacted (e.g., infected) with one or more viral vectors.

### Decreasing Salt Concentrations

In some embodiments of any one of the methods disclosed herein, it may be desirable to increase the concentration of KCl present in the medium for a finite period of time, but not for the entire duration of rAAV production. Accordingly, in some embodiments, the concentration of salt (e.g., KCl and/or NaCl) is decreased in a medium (e.g., the producer medium) by either diluting the medium with medium of a lower salt (e.g., KCl and/or NaCl) concentration or changing (e.g., replacing) the medium with one of a lower salt (e.g., KCl and/or NaCl) concentration. In some embodiments, a producer medium that is replaced may be processed to harvest rAAV particles in the medium.

In some embodiments, the diluting or changing the medium occurs between 0.5-10 hours (e.g., 0.5-8, 1-9, 2-6, or 3-4 hours) after the time at which the mammalian cells are contacted (e.g., infected) with the one or more viral vectors, or after salt (e.g., KCl and/or NaCl) concentration in the medium has been increased (e.g., by supplementation). In some embodiments, the diluting or changing the medium occurs between 0.5 and 8 hours after the time at which the mammalian cells are contacted (e.g., infected) with the one or more viral vectors.

### Cells and Culture Formats

It should be appreciated that any cell or cell line that is known in the art to produce rAAV particles can be used in any one of the methods disclosed herein. In some embodiments, a cell used to produce rAAV in any one of the methods disclosed herein is a mammalian cell. Non-limiting examples of mammalian cells that can be used to produce or package rAAV are HEK293 cells, COS cells, HeLa cells, HeLaS3, BHK cells, CHO cells or PER.C6® (see, e.g., ATCC® CRL-1573™, ATCC® CRL-1651™, ATCC® CRL-1650™, ATCC® CCL-2, ATCC® CCL-2.2, ATCC® CCL-10™, or ATCC® CCL-61™).

AAV (e.g., rAAV) that are used for research and clinical applications are commonly produced in human embryonic kidney cells (e.g., HEK293 cells) by infection, wherein the cells are grown on an adherent flat platform (e.g., culture vessels like T225 or CellSTACK® or HYPERStack® flasks). In some embodiments, cells are adapted to be cultured in suspension culture. Such cells are referred to herein as “suspension adapted” cells. Methods of adapting adherent cells for suspension culture are known in the art. Aspects of the disclosure thus relate to methods of AAV production using suspension adapted cells (e.g., suspension adapted HEK293 cells).

In some embodiments, the mammalian cells that are used to produce rAAV according to the present disclosure are suspension adapted cells. In some embodiments, the suspension adapted cells are suspension adapted HEK293 cells. In some embodiments of any of the methods disclosed herein, suspension-adapted HEK293 cells are derived from adherent HEK293 cells. In some embodiments, suspension-adapted HEK293 cells are obtained from a commercial source. In some embodiments, suspension-adapted cells are cultured in a shaker flask, a spinner flask, a cell bag, or a bioreactor.

In some embodiments, suspension-adapted HEK293 cells are highly permissive to HSV infection, and can be used for the production of infectious AAV particles using HSV infection protocols. In some embodiments, protocols for large scale AAV manufacturing can be based on using suspension-adapted HEK293, for example to generate high yields of potent vector particles. The production of high titer, highly potent rAAV preparations provides significant advantages over currently available protocols and supports large scale clinical research that benefits patients worldwide.

In some embodiments, a unique advantage using HSV infection in combination with the HEK293 suspension platform is the high quality of the virus generated. The potency of the virus generated from the HSV/HEK293 combination is significantly higher than those produced by infection of suspension BHK cells. Other methodologies have failed to provide any advantages related to vector potency. In contrast, the present suspension format allows for large-scale, high titer, and high potency viral (e.g., rAAV) manufacturing. This allows for comprehensive and exhaustive pre-clinical and clinical studies, and can have a significant impact on the many genetic diseases in need of efficient therapeutic protocols. In some embodiments, the present suspension format also allows for bulk manufacturing (e.g., of rAAV) in short time periods using low amounts of human manipulation, thereby reducing the cost of manufacturing and the resulting cost of production (e.g., of a therapeutic rAAV).

As described herein, inventors of this disclosure have discovered that salt (e.g., KCl) is a critical component of an inoculum that improves yields in the production of rAAV in suspension-adapted cells, but that is not adjusted routinely. However, it should be appreciated that methods provided herein can be advantageous in the production of rAAV in adherent cells as well. Accordingly, cells in any one of the methods described herein may be cultured in adherent format (e.g., in cell culture dishes or T-flasks, or a multilayer tissue culture flask such as a Corning CellSTACK®, Corning HYPERstack® or Nunc EasyFill™ Cell Factory) or in suspension format (e.g., in a bioreactor such as a Wave bioreactor, a shaker flask, a spinner flask or a cellbag). In some embodiments, cells are attached to a substrate (e.g., microcarriers) that are themselves in suspension in a medium.

Infection of Cells to Introduce AAV Rep and Cap Proteins and/or One or More Genes of Interest

There are numerous methods by which AAV rep and cap genes, other AAV helper genes, and one or more genes of interest can be introduced into cells to produce or package rAAV. In some embodiments of any one of the rAAV production methods disclosed herein, AAV rep and cap genes and one or more genes of interest can be introduced into cells by infection of viral vectors harboring AAV rep and cap genes and one or more genes of interest.

AAV rep and cap genes may be harbored by one or even more than one (e.g., two or three) vectors (e.g., viral vectors). Similarly, more than one (e.g., two or three) gene of interest (e.g., a gene encoding a therapeutic protein) may be harbored by one or even more than one (e.g., two or three) vector (e.g., viral vectors).

In some embodiments, the one or more viral vectors are a retroviral vector, a lentiviral vector, a baculovirus vector, and/or an adenoviral vector. In some embodiments, one or more viral vectors are a recombinant herpes simplex virus vector (rHSV vector) or an Epstein Barr virus vector. In some embodiments, helper genes other than rep and cap genes are harbored on vectors other than the ones harboring rep and cap genes or a gene of interest.

Methods and compositions for generating viral vectors (e.g., rHSV vectors) with AAV rep and cap genes and/or one or more genes of interest are known in the art (see, for example, Aponte-Ubillus, et al. (2018), Appl. Microbiol. Biotechnol., 102(3): 1045-54; and U.S. Pat. No. 7,091,029, the contents of which are incorporated herein by reference).

It is to be understood that any combination of vectors can be used to introduce AAV rep and cap genes and one or more genes of interest to a cell in which rAAV particles are to be produced or packaged. For example, in some embodiments a first rHSV encoding a gene of interest flanked by AAV inverted terminal repeats (ITRs), and a second rHSV encoding AAV rep and cap genes can be used. In some embodiments, the first and second rHSVs are contacted to cells together (e.g., simultaneously). In some embodiments, one or more helper genes is constitutively expressed by the cells and does not need to be infected into the cells (e.g., by contacting the cells with one or more viral vectors).

In some embodiments, an AAV rep gene encodes rep proteins Rep78, Rep68, Rep52, and Rep40. In some embodiments, an AAV cap gene encodes capsid proteins VP1, VP2, and VP3. In some embodiments, an AAV cap gene encodes an assembly activating protein (AAP) and/or a membrane-associated accessory protein (MAAP).

It should be appreciated that AAV rep, cap, and other helper genes (e.g., E1a gene, E1b gene, E4 gene, E2a gene, or VA gene) can be of any AAV serotype. Similarly, AAV ITRs can also be of any AAV serotype. For example, in some embodiments, the AAV ITRs of any one of the methods disclosed herein are AAV-2 ITRs. In some embodiments, AAV ITRs are from AAV-1, AAV-3, AAV-4, AAV-5, AAV-6, AAV-7, AAV-8, AAV-9, AAV-10, AAV-11, AAV-12, or any other AAV serotype (e.g., a hybrid serotype harboring sequences from more than one serotype).

In some embodiments, AAV cap gene is an AAV-9, AAV-2 or AAV-5 cap gene. In some embodiments, an AAV cap gene is from AAV-1, AAV-3, AAV-4, AAV-5, AAV-6, AAV-7, AAV-8, AAV-9, AAV-10, AAV-11, AAV-12, or any other AAV serotype (e.g., a hybrid serotype harboring sequences from more than one serotype). In some embodiments, the AAV rep and cap genes for the production of a rAAV particle are from different serotypes. For example, the rep gene can be from AAV-2 whereas the cap gene can be from AAV-9.

In some embodiments, the AAV rep or cap genes are operably linked to a promoter. In some embodiments, a gene of interest (e.g., encoding a therapeutic agent) is operably linked to a promoter. A promoter may be constitutive or inducible, and may be naturally-occurring or synthetic. Non-limiting examples of constitutive viral promoters include the Herpes Simplex virus (HSV), thymidine kinase (TK), Rous Sarcoma Virus (RSV), Simian Virus 40 (SV40), Mouse Mammary Tumor Virus (MMTV), Ad E1A, and cytomegalovirus (CMV) promoters. Non-limiting examples of constitutive mammalian promoters include various housekeeping gene promoters, as exemplified by the β-actin promoter (e.g., chicken β-actin promoter), and human elongation factor-1α (EF-1α) promoter. Non-limiting examples of suitable inducible promoters include those from genes such as cytochrome P450 genes, heat shock protein genes, metallothionein genes, and hormone-inducible genes, such as the estrogen gene promoter. Another example of an inducible promoter is the tetVP16 promoter that is responsive to tetracycline. Synthetic promoters are also contemplated herein. A synthetic promoter may comprise, for example, regions of known promoters, regulatory elements, transcription factor binding sites, enhancer elements, repressor elements, and the like.

In some embodiments, a gene of interest encodes a marker protein (e.g., GFP). In some embodiments, a gene of interest encodes a therapeutic agent. In some embodiments, the therapeutic agent is a polypeptide, a peptide, an antibody (or an antigen-binding fragment thereof), a ribozyme, a peptide-nucleic acid, an siRNA, an RNAi, an antisense oligonucleotide, or an antisense polynucleotide. In some embodiments, a gene of interest encodes an RNA. In some embodiments, the RNA is a regulatory RNA, such as an siRNA, or another regulatory RNA (e.g., an RNA which can be therapeutically useful). In some embodiments, a gene of interest encodes an enzyme, hormone, antibody, receptor, ligand, or other protein. In some embodiments, a gene of interest encodes a therapeutically useful protein. In some embodiments, the therapeutically useful protein is therapeutic for lysosomal storage disease. In some embodiments, the therapeutically useful protein is therapeutic for a muscular disability, myopathy, or cardiomyopathy.

In some embodiments, a composition comprising an rAAV described herein can be used to treat a mammalian subject (e.g., a human). In some embodiments, the mammalian subject has or has had cancer, diabetes, autoimmune disease, kidney disease, cardiovascular disease, pancreatic disease, intestinal disease, liver disease, neurological disease, neuromuscular disease, Batten disease, Alzheimer's disease, Huntington disease, Parkinson's disease, pulmonary disease, an α-1 antitrypsin deficiency, a neurological disability, a neuromotor deficit, a neuroskeletal impairment, an ischemia, a stroke, a lysosomal storage disease, Pompe disease, Duchenne Muscular Dystrophy, Friedreich's Ataxia, Canavan disease, an aromatic L-amino acid decarboxylase deficiency, Hemophilia A/B, or any other disease, or any combination thereof.

A Method for Improving the Quality of rAAV Preparations

The quality of an rAAV preparation can be assessed using various parameters, for example the yield of rAAV particles, concentration of rAAV particles, infectivity of the rAAV particles, the percentage of particles that carry the gene of interest (or lack of empty capsids), or contamination by bacterial or fungal elements, contamination by either proteins from the cells used to prepare rAAV (e.g., HEK293 cells) or from a virus used to infect the cells used to prepare rAAV (e.g., HSV to introduce rep or cap protein to the cells), or contamination from either genomic DNA from the cells used to prepare rAAV (e.g., HEK293 cells) or from a virus used to infect the cells used to prepare rAAV (e.g., HSV to introduce rep or cap protein to the cells). Another factor by which the quality of an rAAV preparation is assessed is the degradation of rAAV particles. Degradation of an rAAV can be analyzed using various methods known in the art to determine the size of rAAV particle proteins. For example, proteins in a preparation of rAAV can be run on an SDS gel and interrogated for size. Chromatography or light scattering techniques may also be used to determine size of proteins and particles. Additionally, the modification at the AAV capsid surface that can occur within the cell in which the rAAV is produced, or in the media it is released in during production or purification, can be used to assess quality. Such modifications are generally called post translational modifications (PTMs), and have been shown to depend, at least in part, on the production system (e.g., transfection versus baculovirus), the purification method, and/or the storage methods. These modifications have been shown to contribute, at least in part, to loss of rAAV quality, notably to loss of infectivity both in vitro and in vivo models.

In some embodiments, quality of an rAAV preparation is improved by infecting the cells used for rAAV production rather than transfecting them with necessary AAV genes (e.g., rep, cap and helper genes). In some embodiments, quality of an rAAV preparation is improved by supplementing with KCl the medium in which cells produce rAAV. In some embodiments, quality of an rAAV preparation is improved by supplementing with KCl the medium in which cells are contacted with one or more viral vectors (e.g., infected), whether the cells are cultured in adherent form or in suspension.

Other Parameters Influencing rAAV Production and Yield

It is to be understood that any one of the methods involving increasing KCl concentration in media and using suspension-adapted HEK293 cells for rAAV production can be optimized for other parameters. Non-limiting examples of such parameters are multiplicity of infection (MOI) during infection, cell density, shaker speed of suspension cultures, and media pH at different stages.

### EXAMPLES

### Example 1. Production of AAV9-GFP in Suspension-Adapted HEK293 Cells

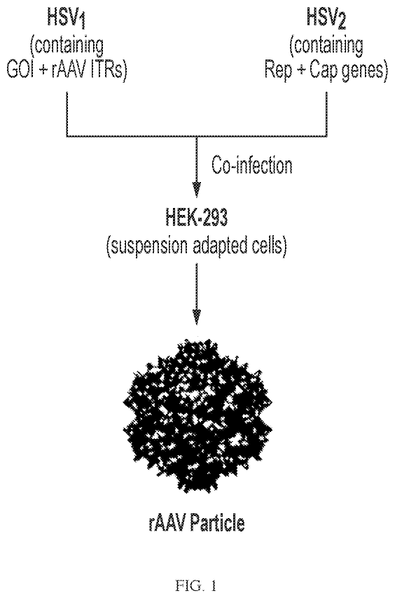

Cell lines described in Table 1 were contacted (e.g., co-infected) with first and second rHSVs as described herein (FIG. 1). FIG. 1 shows one embodiment of how rAAV particles are produced as disclosed herein. Two HSVs, one containing a gene of interest (GOI) between rAAV ITRs, and the other containing rep and cap genes, are used to co-infect HEK-293 cells that are adapted for suspension culture. After the cells package the GOI in rAAV particles, particles are harvested and purified. Reagents and Materials described in Tables 2 and 3, respectively, were used.

Approximately 1E8 cells were resuspended in fresh medium at 1E6 c/mL on the day of infection. Different cell lines and medium combinations were used. After coinfection with the rHSV stocks, the cells were harvested by centrifugation and virus was released by a series of 3 freeze/thaws in lysis buffer (Tris, NaCl, pH 8.5). Lysates were endonuclease (e.g., Benzonase®) treated for 30 minutes at 37° C. and clarified by centrifugation. Infectious titers were assayed by green cell assay (also known as transduction assay): C12 cells were infected by serial dilution of the AAV9-containing crude lysates and wtAd5. GFP-expressing cells were counted after 48 hours and transducing titers were calculated based on the dilution used.

Four different cell conditions were compared (s293-VC-suspension adapted: suspension adapted HEK293 cells grown in Joklik medium containing 5% FBS and 1% AA; s293-VC Expi: suspension adapted HEK293 grown in serum-free medium; EXPI293F™ cells grown in serum-free EXPI293 medium; and BHK21 cells grown in DMEM 5% serum, 1% AA).

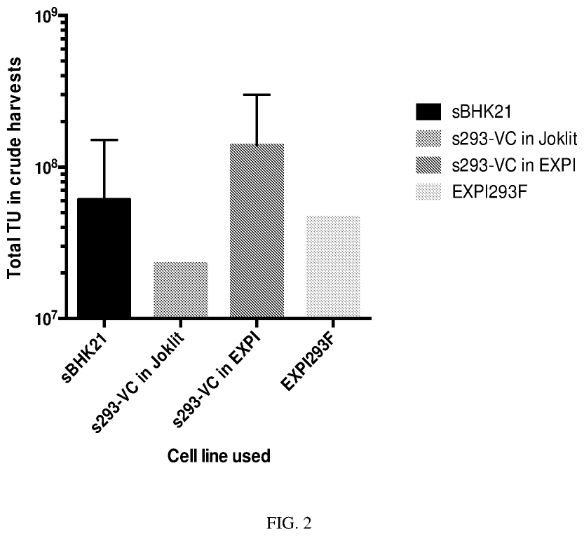

Production of rAAV (AAV9-GFP, expressing GFP) was evaluated and the results are shown in Table 4 and FIG. 2.

FIG. 2 shows production of rAAV particles comprising GFP in different cell types. Suspension adapted EXPI293F™ cells and suspension adapted BHK21 cells were obtained from commercial vendors. Suspension adapted s293-VC were generated in the laboratory from adherent HEK293. EXPI293F™ were grown in serum-free EXPI293 medium. BHK21 were grown in DMEM, 5% serum, and 1% antibiotic/antimycotic (AA). s293-VC were grown in Joklik medium containing 5% FBS and 1% AA, or in serum-free EXPI293 medium. Cell lines were co-infected with first and second rHSV as described herein. Approximately 1E8 cells were resuspended in fresh medium at 1E6 c/mL on the day of infection. Cells were harvested 48 hours post-infection by centrifugation and virus was released by a series of three freeze/thaws in lysis buffer (Tris, NaCl, pH 8.5). Lysates were submitted to endonuclease (e.g., Benzonase®) digestion and clarified by centrifugation. Infectious titers were assayed by green cell assay (also known as transduction assay). C12 cells were infected by serial dilution of the AAV9-containing crude lysates and wtAd5. GFP-expressing cells were counted under the microscope after 48 hours and transducing titers were calculated based on the dilution used. Total transducing units (TU) are shown, demonstrating that it is feasible to use HEK293 cells that were freshly adapted in the laboratory, as compared to commercially available cell lines.

TABLE 1
Cell lines

| Cell lines | Vendor | Catalog number |
|---|---|---|
| s293-VC - suspension | N/A | N/A |
| adapted | | |
| S293-VC Expi - serum free | N/A | N/A |
| adapted | | |
| EXPI293F ™ | Life Technologies | A14527 |
| BHK21 C13-2P | Sigma | 84111301-1VL |

TABLE 2
Reagents

| SMEM Joklik's modification | Lonza | 04-719Q (1 L) |
|---|---|---|
| EXPI293 Expression | Gibco - Life | A14351-01 (1 L) |
| medium | Technologies | |
| SFM4Transfx-293 | Hyclone | SH30860.02 |
| Fetal Bovine Serum | Corning | |
| Dulbecco's Modifications | Corning - Cellgro | 10-017-CM (1 L) |
| of Eagle's Medium | | |
| Antibiotic/Antimycotic | Gibco - Life | 15240-062 (100 mL) |
| | Technologies | |

TABLE 3
Materials

| Reagent | Vendor | Catalog number |
|---|---|---|
| 125 mL Spinner Flask | Corning | 3152 |
| 500 mL Spinner Flask | Corning | 3153 |
| 125 ml Shaker Flask | Corning | 431143 |
| 500 ml Shaker Flask | Corning | 431145 |
| 2000 ml Shaker Flask | Corning | 431255 |
| 5000 ml Shaker Flask | Corning | 431685 |
| Magnetic stirrer | Wheaton | Model # W900701---A |
| Incubator | Infors-HT | I10002P |
| Incubator | Forma | Model # |

TABLE 4
Results

| | | | | Number of |
|---|---|---|---|---|
| Cell lines | Average | SDEV | % CV | experiments |
| BHK21 | 6.08E7 | 9.03E7 | 149 | 16 |
| s293-VC | 2.3E7 | N/A | N/A | 1 |
| s293---VC/Expi | 1.39E8 | 1.61E8 | 116 | 5 |
| EXPI293F ™ | 4.6E7 | N/A | N/A | 1 |

### The Production System Using EXPI293™ is Highly Scalable

The next aim was to evaluate the scalability of this novel suspension production procedure by evaluating total viral production in crude lysates. Analyzing Benzonase®-treated crude lysates was critical to assess total production capabilities independently of the purification processes that may vary across laboratories and even across the different AAV serotypes.

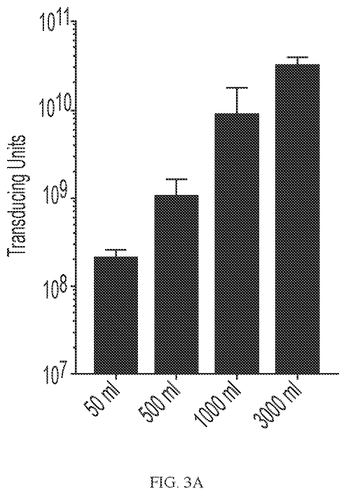

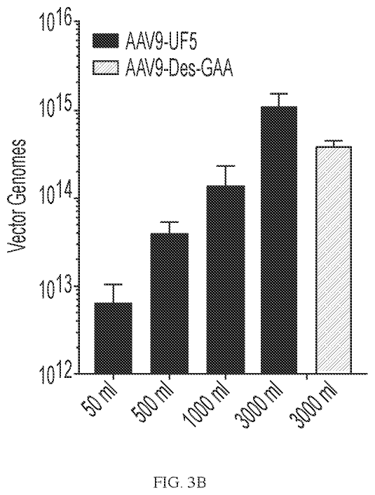

FIGS. 3A-3C show the total transducing unit and vector genome yields as a function of working production volumes comprising between 50 mL and 3 L, or a 60-fold scale-up. Transducing units for rAAV9-GFP (FIG. 3A) and total vector genomes for rAAV9-GFP and rAAV9-Des-GAA (FIG. 3B) were assessed in crude lysates from EXPI293F™ cultures ranging from 50 to 3,000 mL. The increase in yield was relatively linear to the volume increase for scales up to 0.5 L with an average of approximately 7-fold increase in transducing units (TU) when compared to 50 mL (FIG. 3A). However, the yield increase was about twice as much as the increase in volume from 50 mL to 1 L (˜40-fold) and to 3 L (˜155-fold). FIG. 3C shows total vector genome values represented as vector genomes per cell at each scale. The highest yields were obtained from 3 L working volumes for rAAV-GFP with an average of 1.24×10¹⁵ vg±4.66×10¹⁴ (n=4) or 4.12×10¹⁴ vg/L±1.55×10¹⁴ in crude lysates. This resulted in an average of 4.12×10⁵ vg/cell±1.55×10⁵ (FIG. 3C). The increase in infectious units paralleled the particle titers, with an average of 3.16×10¹⁰±8.90×10⁹ TU (transducing units) for the 3 L crudes as compared to 1.05×10⁹±5.74×10⁸ (n=4) for 0.5 L, an ˜30-fold increase. Yields for rAAV9-GAA were more consistent with expected scaled-up increase with an average of 3.97×10¹⁴ total vector genomes per 3 L or 1.2×10¹⁴ vector genomes per liter culture (n=2; FIG. 3B). A better growth environment in the 5 L flask, utilized to perform the 3 L working production batches, may explain this more favorable scale. The ratio of medium to the flask volume, the shaking speed and/or the flask shape may promote oxygenation, reduce cell shear or improve virus/cell contact. As observed at smaller scales, the infectivity of the rAAV9 batches was maintained throughout the scaling process with a ratio VG:TU of 3.88×10⁴±1.88×10⁴ (11 independent runs at 0.5, 1 and 3 L).

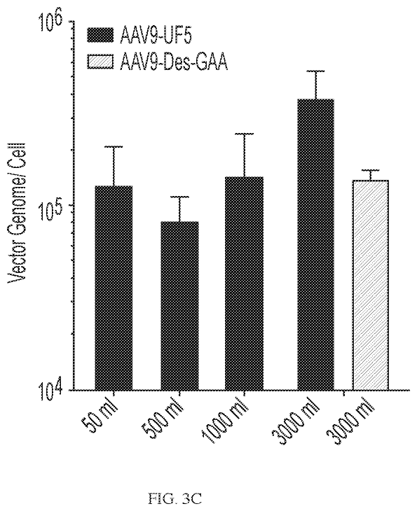

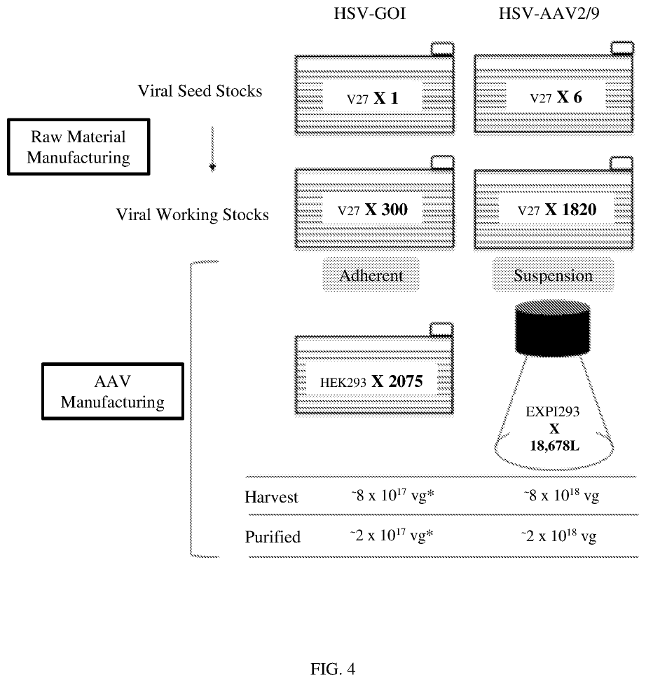

FIG. 4 shows the theoretical scalability of the HSV system. Extrapolation was based on the current average rHSV yields for the constructs. HSV stocks were produced from adherent V27 cells grown in 10-layer flasks (CS10®). The number of production units (CS10® for adherent or liter for suspension) and cell type (V27, HEK293, or Expi293F™) are indicated for each step and were rounded for clarity. HSV-GOI and HSV-Rep2Cap9 seed stocks were produced from 1 and 6 CS10®, respectively, to support MOIs used in the adherent platform (2:12, respectively).

Salt Supplementation is a Strategy to Boost rAAV Production that Works for Different AAV Serotypes and Genes to be Packaged and at Different Scales.

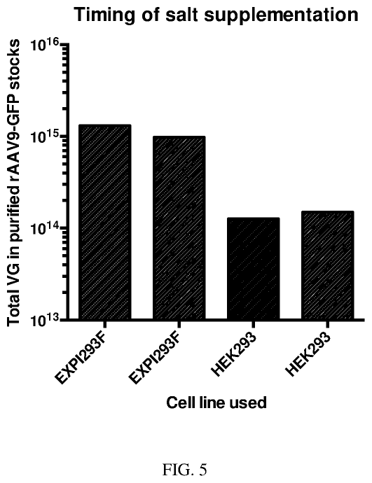

FIG. 5 shows that salt supplementation can occur 6 hours (gray-lined bars) after HSV co-infection in both adherent format (HEK293) or suspension format (EXPI293F™) without affecting the overall yield (black bars indicate that salt was added at the time of HSV coinfection) as measured by the total vector genomes. Sodium chloride (NaCl) is exemplified. HEK293 were infected at MOI 2:12 and EXPI293F™ cells were infected at MOI 2:4 and harvested after 48 hours. AAV9 particles were purified and stocks titered for vector genomes. Also noted is that simultaneous coinfection is not required for optimal AAV production (FIG. 6). HSV-GFP can be added up to 4.5 hours (270 min) after HSV-AAV9 has been added to the production medium without negatively impacting the overall AAV yield, as measured by total transducing units in EXPI293F crude harvests.

### Example 2: Log Increase in AAV Titer is Specifically Mediated by Sodium and Potassium Chloride

### Materials and Methods

Cell lines. Expi293F™ (ThermoScientific, Waltham, MA) were maintained in Expi293F™ media and grown in shaker incubators (Multitron, Infors HT) at 5% CO₂, 37° C., 125 RPM and 75% humidity. Cell concentration was maintained between approximately 5×10⁵ c/mL and 5×10⁶ c/mL during passaging. Cell counts, viability, and size were assessed using Countess™ II Automated Cell Counter (ThermoFisher Scientific). For all production experiments, a cell viability superior to 90% was required. C12 cells (originally obtained from Dr. P. Johnson, Children's Hospital of Philadelphia, PA) were maintained in DMEM supplemented with 5% FBS and 50 μm/mL Geneticin (Sigma, St. Louis, MO).

Plasmids. Plasmids pTR-UFS, pDG-UF9-KanR, and pMA-P5-Rep2Cap9 were previously described in Adamson-Small, et al. (Mol Ther Methods Clin Dev. 2016; 3: 16031). pTR-UF5 contains an AAV2-ITR vector genome cassette for the expression of humanized green-fluorescent protein (hGFP) under the cytomegalovirus promoter (CMV), identical to the rAAV genome present in rHSV-GFP. Plasmid pDG-UF9-KanR contains the AAV2 rep and AAV9 capsid ORFs under the MMTV and AAV2 P19/P40 promoters and the Ad5 helper genes as described in Grimm, D., et al., (1998), Hum Gene Ther 9: 2745-2760. Plasmid pMA-P5-Rep2Cap9 contains the endogenous AAV2 Rep ORF with p5, p19, and p40 promoters, and AAV9 capsid sequence.

rHSV. Production of recombinant herpes viruses rHSV-GFP and rHSV-AAV9 was performed as previously described in Adamson-Small, et al. (Mol Ther Methods Clin Dev. 2016; 3: 16031). Briefly, a full genome of recombinant AAV2 consisting of the AAV2 ITR, the CMV promoter, and the humanized GFP sequences were inserted in the genome of HSV type 1 deletion mutant d27.1 to create rHSV-GFP. HSV stocks were titered by Plaque Assay as described in Adamson-Small, et al. (2016). Helper recombinant HSV-AAV9 contained the entire expression cassette of AAV2 Rep and AAV9 Cap, under their respective endogenous promoters, as described in Adamson-Small, et al. (2016).

Small-scale AAV production. Expi293F™ cells were seeded in 125 mL Corning® Erlenmeyer cell culture flasks with vented cap (Corning Millipore-Sigma) at 1×10⁶ cells/mL in 25 or 50 mL final culture volumes. Cells were co-infected within 1 hour at a multiplicity of infection (MOI) of 2:4 with rHSV-GFP and rHSV-AAV9, respectively. For media supplementation experiments, the inoculum containing rHSV, cell media, and defined salt or glucose concentrations were mixed and immediately added to the cell-containing flasks. Supplementing reagents were sodium chloride solution (5 M NaCl; AccuGene® Lonza) potassium chloride solution (2 M KCl; ThermoFisher Scientific), Dulbecco's phosphate buffer saline with calcium and magnesium solution (10× DPBS; Corning, Cellgro), zinc chloride solution (0.1 M solution; Millipore Sigma, St. Louis, MO), magnesium chloride solution (1 M MgCl₂; ThermoFisher Scientific), calcium chloride solution (Millipore Sigma, St. Louis, MO), manganese chloride solution (10× Manganese II Chloride; Millipore Sigma, St. Louis, MO), nickel chloride (Nickel II Chloride hexahydrate; powder; Millipore Sigma, St. Louis, MO), sodium selenite (powder; Millipore Sigma, St. Louis, MO), and glucose (200 g/L solution, ThermoFisher Scientific).

Two days after infection, cells were pelleted (2000 RPM, 4° C., 20 min, Sorvall™ ST-ThermoScientific), rinsed in 1×DPBS (Hyclone) and pelleted again. When required, the supernatants were sampled for pH and osmolality and stored at 4° C. Vector genome and infectious titers were assessed in cell crude lysates prepared by a series of three freeze-thaws in 2.5 or 5 mL lysis buffer (50 mM Tris, 150 mM NaCl, pH 8.5), Benzonase® (EMD Millipore, Billerica, MA) digestion (100 units/mL, 30 min, 37° C.), and clarification (3700 RPM, 20 min, 4° C., Sorvall® ST-40R). Crude lysates were stored at −80° C. for the duration of the study.

For timing experiments, cells were co-infected as described above either in the presence or absence of KCl. When co-infected in the presence of KCl, the medium was exchanged after the desired duration (in hours), replaced with fresh medium not containing KCl, and incubated for the remaining of the production period. Alternatively, when co-infected in the absence of KCl, KCl was added at a later time, and either left for the duration for the production period or removed after the desired number of hours and replaced with fresh medium without KCl.

Large-scale production and purification. Large-scale production was performed in 5 L Corning® Erlenmeyer cell culture (Millipore Sigma) flasks with a 3 L production volume and a total of 3×10⁸ cells. Infection and supplementation were performed similarly to the small-scale production, but were grown at 100 RPM. AAV purification was previously described in Adamson-Small, et al. (Mol Ther Methods Clin Dev. 2016; 3: 16031). Briefly, cells were lysed and lysates were digested with Benzonase® (EMD Millipore, Billerica, MA) (crude lysate), clarified by protein flocculation and centrifugation.

Viral particles were purified using a two-step chromatography approach: a cation-exchange step on SP Sepharose Fast Flow (Cytiva/GE Healthcare, Marlborough, MA) was followed by an affinity-based capture using POROSTM CaptureSelect™ AAV9 Affinity resin (ThermoFisher Scientific). The final eluate was formulated in 1×DPBS by dialysis using 10K Slide-A-Lyzer® Dialysis cassettes (3-12 mL, ThermoFisher Scientific). Final concentrated AAV stocks were stored at −80° C.

AAV production by transient transfection. Small-scale transient transfection (50 mL) was performed in EXPI293 cells (5×10⁷ cells in 125 mL shaker flasks) using PEI Max® 40K (Polysciences Inc, Warrington, PA). Plasmids were transfected at equimolar amounts (50 μg total). The salts were added at the time of transfection and left for 72 hours before harvest. For infection and transfection combinations, cells were first transfected for four hours and then infected with rHSV and salts. Cells were harvested after two days for analysis.

Vector genome titer. Vector genome titers were obtained by quantitative real-time PCR (qPCR) as previously described Adamson-Small, et al. (Hum Gene Ther Methods. 2017 Feb. 1; 28(1): 1-14), using primers UF5-3F (5′-CCAGGTCCACTTCGCATATT; SEQ ID NO: 1) and UF5-3R (5′-GCGTGCAATCCATCTTGTTC; SEQ ID NO: 2) and plasmid pTR-UF5 for the standard curve.

Transduction assay. Ad5-infected C12 cells were transduced in 96-well plates by serial dilutions of the rAAV-containing samples. After 42-48 hours, GFP-expressing cells were visually counted and the titer was calculated.

pH and Osmolality. Samples were brought to room temperature before analyzing pH and osmolality. pH was measured using the Accumet model AR 15 pH meter with Orion PerpHect ross combination electrode. The pH meter was calibrated with US standard buffers (pH 4 & 7) and the slope was determined to be between 95% to 102% for successful calibration. Osmolality was measured using a Precision Touch Micro OSMETTE 6002 osmometer according to the manufacturer's protocol. The osmometer was calibrated with calibration buffers (100, 500, & 1500 mOsm/kg) before analyzing samples. Special tubes (Cat #2038) provided by Precision systems were used for analyzing osmolality.

Statistical analysis. GraphPad Prism software (GraphPad; La Jolla, CA) was used to analyze differences between groups utilizing a one-way ANOVA followed by Dunnett's post-test for group comparison. P-values<0.05 were considered statistically significant.

### Results

### Potassium Chloride Significantly Increases Volumetric and Specific AAV Yields.

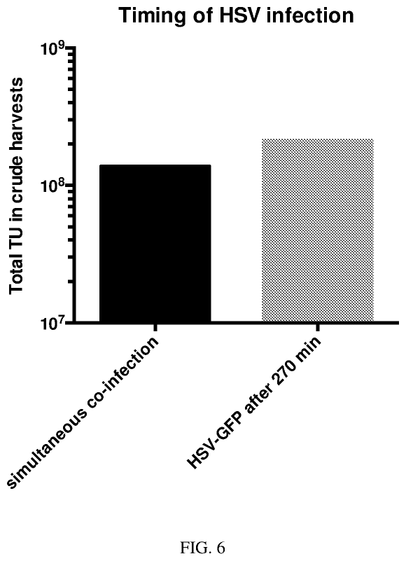

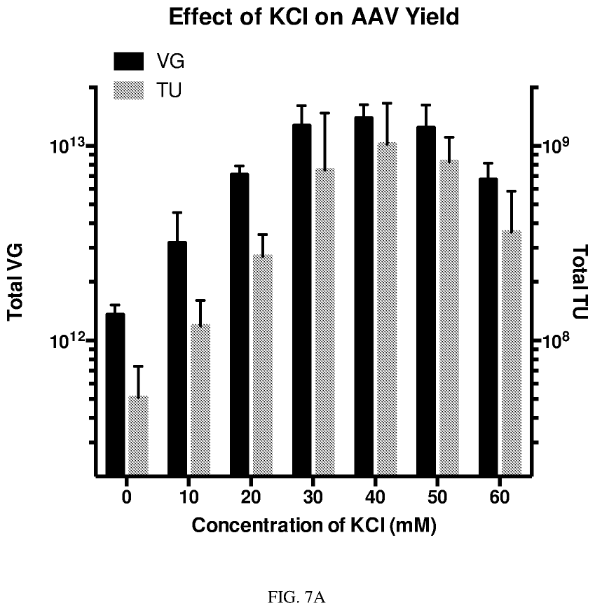

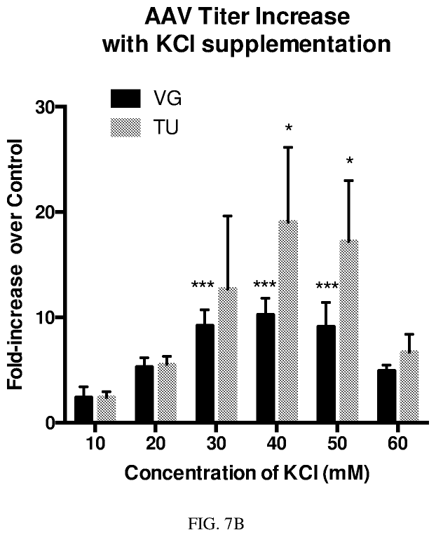

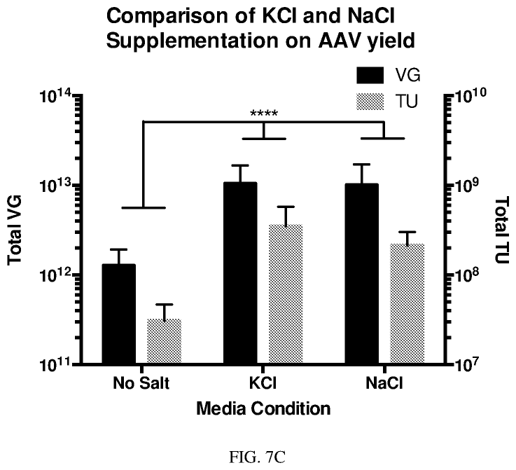

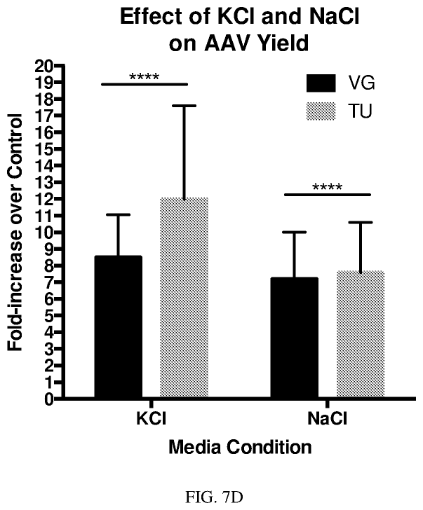

A series of salts were tested to evaluate their effect on AAV yield. These salts included potassium chloride (KCl; tested between 10 and 60 mM), zinc chloride (ZnCl₂), magnesium chloride (MgCl₂), calcium chloride (CaCl₂), manganese chloride (MnCl₂), and cobalt chloride (CoCl₂). Salts were added at various concentrations directly to the production medium and concurrently with the addition of rHSV-GFP and rHSV-AAV2/9. FIG. 7A shows total vector genome particles (VG; black bars) and transducing units (TU; gray bars) assessed in the Benzonase-treated crude lysates generated from the various conditions after 48 hours in cells infected with rHSV-GFP and rHSV-AAV2/9 in the presence of various concentrations of KCl (50 mL production volume, 5×10⁷ cells, n=3). Averages, standard deviations, and number of independent experiments (n) are shown throughout FIG. 7.

It was observed that, among all the salts tested, KCl had the most significant effect on AAV production, and the optimal concentration range was between 30 and 50 mM KCl (FIG. 7A), but with an effect starting at as low as 10 mM. Concentrations above 50 mM resulted in a decline of the rate increase. FIG. 7B shows vector genome and infectious titers from FIG. 7A (n=3) expressed as fold-increase ratio of KCl-treated conditions versus non-treated controls. The average increase in vector genome titer was 10-fold when compared to the non-KCl-treated condition at 40 mM (FIGS. 7A and 7B). Importantly, the increase in particle titers was strongly correlated with an increase in transducing unit (TU) titers, with an average of a 20-fold increase in TU titers at 40 mM (see FIGS. 7A and 7B) over the control conditions.

Among all the other salts tested, only zinc chloride, tested between 0.01 to 16 mM, had a minor effect of approximately 2-fold increase at 0.2 mM. This result was not statistically significant, and ZnCl₂ was not studied further (data not shown). All the other salts tested, magnesium chloride (0.5 to 40 mM), calcium chloride (0.02 nM to 2 mM), cobalt chloride (25 to 100 μM), sodium selenite (5 nM to 20 μM), and iron nitrate (1 nM to 10 mM) had either no effect or led to a reduction in the AAV titer at the concentration range tested. At some concentrations, CaCl₂ and ZnCl₂ triggered the formation of a precipitate in the culture medium, which could be responsible for the loss in AAV.

The effect of KCl and NaCl was then directly compared in side-by-side experiments, with KCl supplemented at 40 mM and NaC1 at 60 mM, as previously established in Adamson-Small, et al. (Hum Gene Ther Methods. 2017 Feb. 1; 28(1): 1-14). Because the HSV stocks were formulated in a production medium that contained 600 mM NaCl, the final concentration of NaCl added to the production medium was adjusted to take into account the amount of each rHSV added to the production, which varied depending on each HSV stock plaque forming unit's titer. FIG. 7C shows vector genome and transducing unit titers from KCl (40 mM, n=37) or NaCl (60 mM, n=19) supplemented conditions, versus control (no salt; n=37). **** p<The average vector genome yield was 1.04×10¹³±6.20×10¹² with KCl supplementation, and 1.01×10¹³±6.99×10¹² vg with NaCl supplementation, as compared to 1.27×10¹² vg±6.52×10¹¹ without salt supplementation (FIG. 7C). Despite the variability in titers across the numerous experiments, the differences were found to be statistically highly significant both for vg and tu titers.

FIG. 7D shows the mean fold-increase of KCl- or NaCl-treated conditions versus a non-treated (e.g., non-supplemented) control (same experiments as shown in FIG. 7C). **** p<0.0001. The average particle yield increase was 8.50±2.56 fold for KCl and 7.19±2.82 fold increase with NaCl. This increase was smaller than the ones previously observed with NaCl alone Adamson-Small, et al. (2017). As noted earlier, the vector genome yield was highly correlated with the transducing unit yield, with an average increase in transducing units of 11.97- and 7.58-fold increases for KCl and NaCl, respectively (FIGS. 7C and 7D). This correlation resulted in reduced vg:tu ratios, a measure often used to express AAV potency, with lower values reflecting a potential increase in potency, at least when assessed in vitro.

To verify that the observed vector genome titer increase was not due to the presence of possible traces of KCl in the cell crude lysates tested—which could potentially affect the PCR reaction—a mock cell lysate was prepared without HSV/AAV, but with KCl supplementation, and harvested similarly to the AAV-containing samples. This virus-free cell lysate was used to spike in a known amount of a purified AAV stock (e.g., a defined amount or volume of AAV stock was added to the matrix) which was quantified using qPCR. The cell lysate did not impact the overall result of the PCR and the vector genome increase was consistent with an increase in AAV vector genome titer (data not shown). Like for the qPCR assay, it was verified that possible traces of KCl present in the cell lysate did not affect the transduction efficiency of the rAAV in the in vitro assay (data not shown).

Lastly, two 3L-scale, fully purified stocks of rAAV9-GFP were prepared in the presence of either KCl or NaCl supplementation. Both conditions yielded nearly identical titers for both vector genomes and transducing units at each step of the downstream purification procedure (see Table 5). Importantly, the ratio vg:tu was 1.10×10⁴, similar to the ratios obtained in small-scale experiments. This further confirmed that the increase in potency observed in crude lysates was not due to potential impurities in the virus preparations, and that KCl and NaCl triggered a very similar AAV rate increase.

TABLE 5
Comparison of NaCl and KCI supplementation at large-scale production.
AAV9-GFP was produced side-by-side and purified from 3 L scale
production in the presence of NaCl
and KCl. n = 1 each; results expressed in vector genomes (VG).

| | KCl | NaCl |
|---|---|---|
| | supplementation | supplementation |
| Total vector genome (VG) Harvest | 2.77 × 10¹⁵ | 2.54 × 10¹⁵ |
| Total VG Purified Dialyzed Stock | 5.83 × 10¹³ | 4.78 × 10¹³ |
| VG/cell Harvest | 9.23 × 10⁵ | 8.47 × 10⁵ |
| Ratio VG:TU | 1.10 × 10⁴ | 1.10 × 10⁴ |

Combination of NaCl and KCl or Phosphate Buffer Saline also Increases AAV Titer

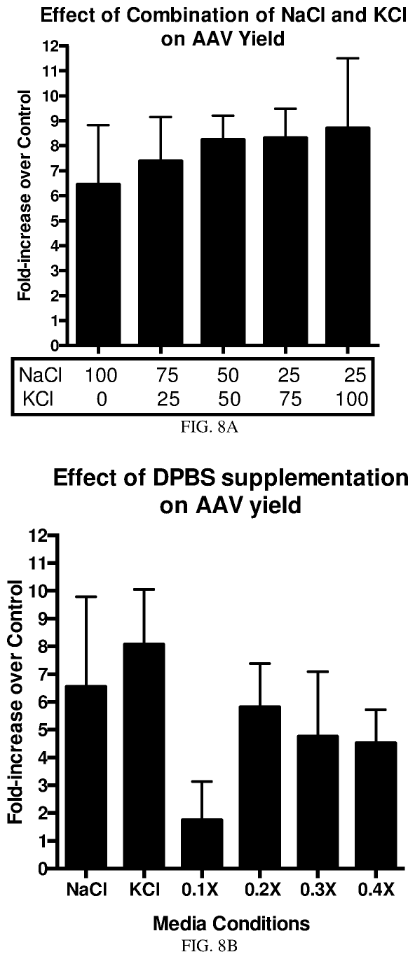

It was next investigated whether the combination of NaCl and KCl could act in synergy to further increase the overall AAV yield. The concentration of NaCl supplemented was varied from 16 mM (only salt from the HSV stocks, or 26.7% of target) to 60 mM (100% of target) and the concentration of KCl was varied from 0 mM (0% of target) to 40 mM (100% of target). One condition had both salts added at 100% of target concentration (e.g., 60 mM NaCl and 40 mM KCl). FIG. 8A shows a combination of NaCl and KCl (n=3). NaCl and KCl concentrations are expressed as percentage of optimal concentration (60 mM and 40 mM, respectively). No significant improvement was observed with any of these conditions when compared to each salt used individually (FIG. 8A), and the condition with both salts added at their optimal concentration (e.g., 60 mM NaCl and 40 mM KCl) resulted in a slight decrease in AAV yield (data not shown). Although the differences were not significant, an upward trend was noted when KCl concentration was increased. This suggested that each salt acted individually in triggering an increase in AAV production, and did not act synergistically.

Lastly, Dulbecco's phosphate-buffered saline (DPBS) was evaluated. DPBS is a commonly used buffer comprising a cocktail of various salts at various concentrations, including NaCl and KCl. Using a 10-fold concentrated DPBS, final concentrations of 0.1× to 0.5× were incorporated into the production medium, and the overall AAV9-GFP yield was evaluated. FIG. 8B shows the effect of Dulbecco's phosphate buffer saline (DPBS) on AAV yield as a fold-increase of DPBS-treated conditions versus control, KCl-treated, and NaCl-treated. (n=3, except for 0.1× and 0.4×, n=2). DPBS concentration is expressed as a factor from 1×. Significant increases were observed at DPBS concentrations of 0.2× and 0.3× (FIG. 8B), but the effect of KCl or NaCl alone (performed side-by-side in the same experiments) was always higher. At 0.2× and 0.3× DPBS, the corresponding concentrations of supplemented NaCl and KCl would be approximately 27 and 41 mM for NaCl and 0.54 and 0.78 mM for KCl. This suggested that DPBS only partially reproduced the effect mediated by NaCl, since the KCl concentration was well below the optimal concentration required for AAV increase. This result also suggested that other compounds present in DPBS do not contribute further to the NaCl-mediated effect, further emphasizing the specificity of the effect to these two salts.

Increase in Titers is Not Correlated with Cell Growth and Infection Rate

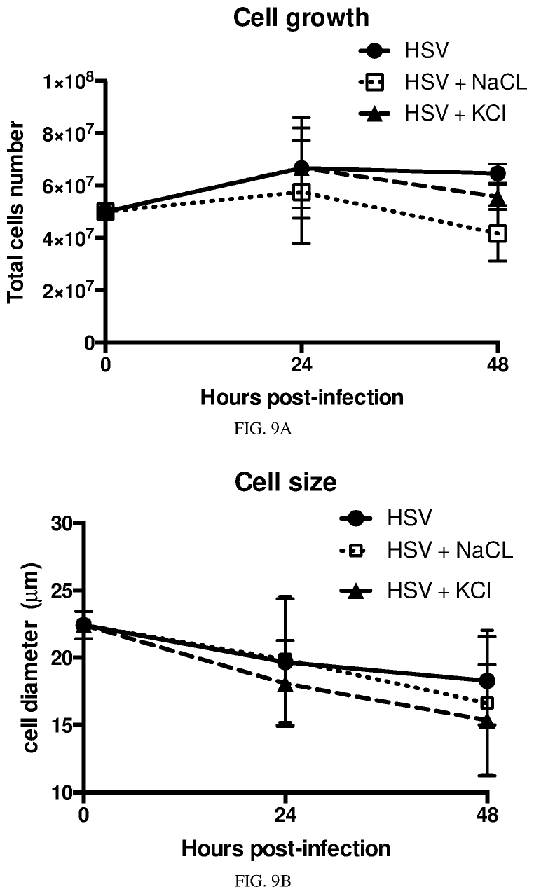

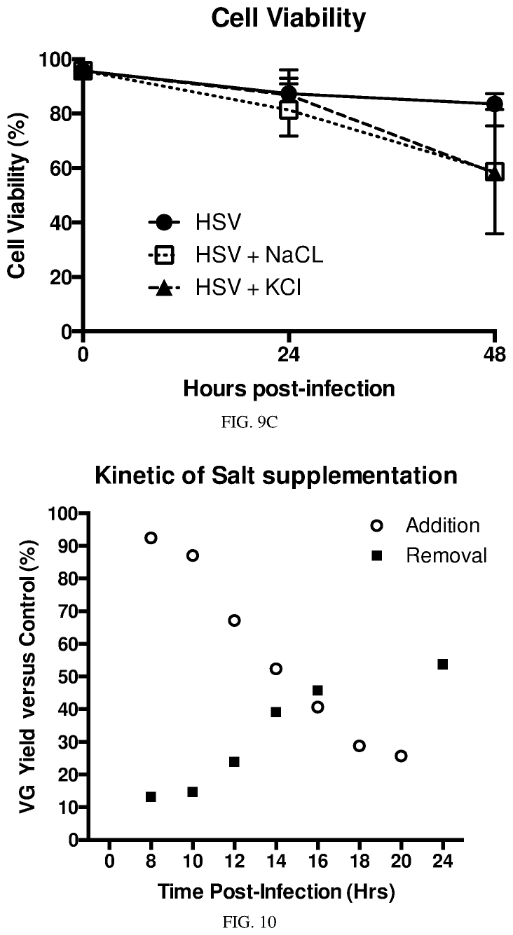

It was next investigated whether the increase in AAV titers, triggered by salt supplementation, could be due to an increase in cell division, size, or viability. FIGS. 9A to 9C show the effect of NaCl and KCl on cell growth, size, and viability during AAV production. Cell number (FIG. 9A), size (FIG. 9B), and viability (FIG. 9C) were assessed at 3 time points during the production cycle. Average and standard deviation shown, n=3. The total number of cells measured at various time points during the course of AAV production was lower for KCl and NaCl-treated conditions, especially at the time of harvest (FIG. 9A). Similarly, cell size (FIG. 9B) and viability (FIG. 9C) were both decreased in the salt-treated conditions. These results were not unexpected, since both HSV and AAV have a cytopathic effect on the producer cells. However, this strongly supports the conclusion that the observed increase in AAV yield was the direct result of a specific yield increase with more AAV produced per cell.

### Effect of Salt Supplementation is Time Sensitive

Previous results indicated that NaCl could be supplemented up to six hours post-infection (Adamson-Small, et al., Hum Gene Ther Methods. 2017 Feb. 1; 28(1): 1-14) while maintaining the same rate increase effect on AAV production. Experiments were performed to further delineate the precise time window within which salt supplementation was optimal to achieve an AAV yield increase (FIG. 10; Addition: one representative experiment for time point hrs post-infection (PI) and one representative experiment for time points 14 to 20 hours PI. Removal: one representative experiment).

In the first set of experiments, salt supplementation was delayed by several hours after the HSV inoculation started (e.g., production time 0). KCl was then supplemented at 8 to 20 hours post-infection (PI). Cells were harvested 48 hours after initiation and vector genome titers were assessed in cell crude lysates. It was observed that KCl supplementation delayed by up to 8-10 hours post-infection could sustain 80-90% of the AAV increase (FIG. 10, open circles). However, further delays (e.g., salt supplementation 12 to 18 hours post-infection) resulted in a sharp decrease in the AAV yield, and no significant effect was observed when the salt was supplemented after 20 hours.

In the next set of experiments, supplementation was carried out at the time of HSV inoculation (e.g., time 0), and the salt was removed at various time points by media exchange (FIG. 10, black squares). After 8 to 20 hours, the medium was removed and replaced with salt-free medium and incubated for the remaining of the production cycle. Vector genome (VG) yields obtained for each time point are expressed as a percentage of the yield obtained in the control condition, which is when KCl is supplemented at the time of co-infection and left for the duration of the production cycle. It was observed that removal of the salt after 8 or up to 12 hours post-inoculation significantly impacted the salt-mediated effect on AAV titers, and that removal of the salt at or after 14 hours sustained ˜40-50% of the total salt-mediated effect. The maximal effect was observed when the salt was left up to the harvest time point. Lastly, salt supplementation was initiated 8- or 10-hours post-infection, and then was subsequently removed 4 or 6 hours later such that the salt exposure encompassed about 6 hours (e.g., between 8-14 or 10-16 hours) from the start of the production. It was found that >65% of the salt-mediated increase was maintained if the salt supplementation was sustained for at least 5-6 hours during the production time window of 8-14 hours (data not shown).

These observations indicate that the salt must be present for between 8-14 hours post-infection to ensure a significant AAV rate increase, and that the first 24 hours post-infection were critical in mediating the maximal effect observed. This confirmed that the original protocol with the salt added at the time of infection was the most optimal, as well as the most suitable and cost-effective in terms of the manufacturing procedure. Altogether this data indicates that the salt mechanism was temporal and transient, and suggests that the effect may be highly correlated with the kinetics of the virus production cycle and/or the cell cycle.

Osmolality of AAV Production Media does not Fully Correlate with AAV Titer Increase Induced by NaCl and KCl

Both NaCl and KCl mediate significant changes in osmolality in a linear manner, and are concentration-based. Accordingly, it was critical to assess whether the AAV rate increase was triggered by or at a specific osmolality value, and whether osmolality alone could be the reason for the observed NaCl- and KCl-mediated effect on AAV.

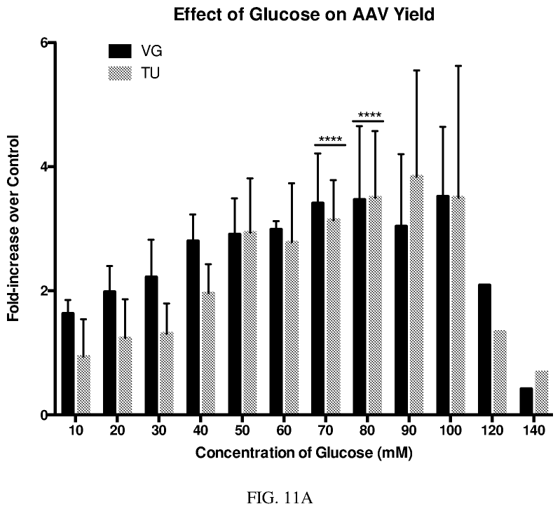

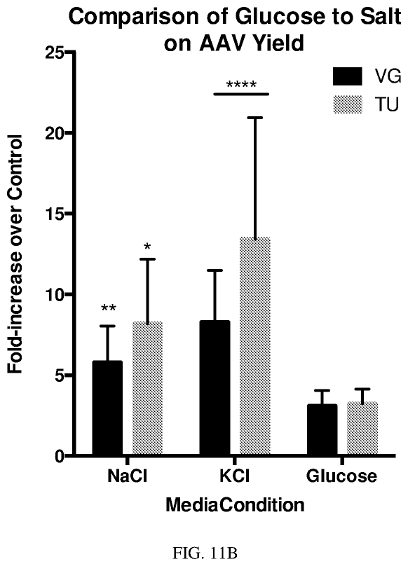

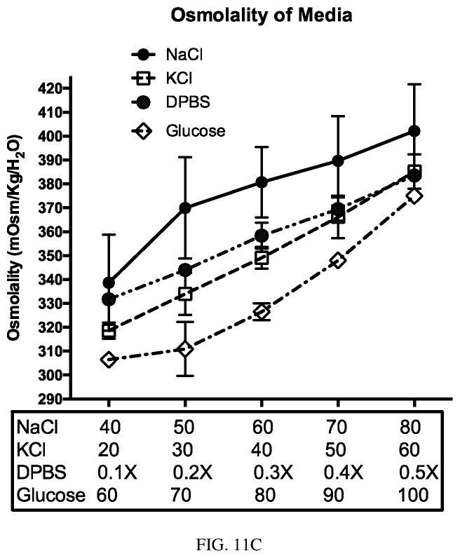

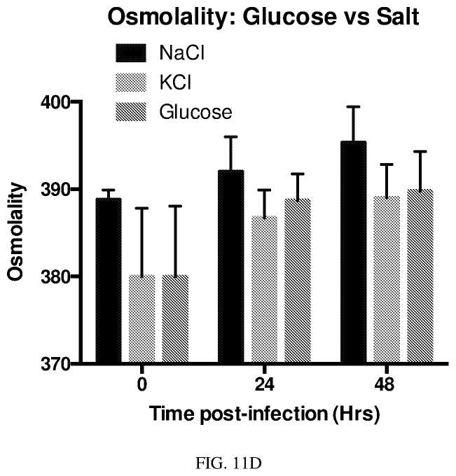

To test this hypothesis, it was first assessed whether another ionic compound like glucose could reproduce the effect observed with KCl and NaCl. Glucose was added at concentrations ranging from 10 to 100 mM, and AAV yields were assessed as previously described. FIG. 11A shows vector genome and infectious titers expressed as fold-increase ratios of treated conditions versus control (non-treated) in the presence of various concentrations of glucose (10, 40, and 50 mM, n=3; 20, 30, and 60 mM, n=4; 70 mM, n=8; 80 and 90 mM, n=9; 100 mM, n=5; 120 and 140 mM, n=1) (averages and standard deviations shown throughout FIG. 11). Interestingly, an increase in AAV titers was observed when glucose was supplemented between approximately 40 and 100 mM, with up to a 3.5-fold increase observed at 70 and 80 mM (FIG. 11A).

FIG. 11B shows the fold-increase ratio of vector genome and transducing units compared NaCl, KCl, or glucose (70 mM) treated conditions versus controls (non-treated) (KCl, glucose, n=7; NaCl, n=4) (* p<0.05; ** p<.001; **** p<0.0001). When directly compared to KCl and NaCl in paired experiments, the glucose-mediated AAV increase was significantly lower than the ones reported with NaCl or KCl (FIG. 11B). Furthermore, and unlike with NaCl and KCl, the observed increase in the transducing unit titers was either similar to or lower than that of the vector genomes. These data suggest that the effect of glucose, although measurable, is not analogous to the observed KCl- and NaCl-mediated effect.

Next, the production medium osmolality values were measured in the presence of NaCl, KCl, DPBS, and glucose. FIG. 11C shows media osmolality mediated by salt and glucose. Osmolality value was measured upon supplementation of the culture medium with various concentrations (mM or fold-dilution) of NaCl, KCl, DPBS, and glucose. It was observed that the osmolality value increased linearly with the concentration of NaCl, KCl, DPBS, and glucose, ranging between 300 and 400 mOsm/kg (FIG. 11C). These values were well above those observed in the non-supplemented culture media, which were 277.44 mOsm/kg ±1.67 (vendor's specifications: 275 to 290 mOsm/kg). The average osmolality value at each compound's optimal concentration (optimal concentrations: NaCl, 60 mM; KCl, 40 mM; DPBS 0.2×; glucose, 80 mM) was approximately: 381 (NaCl); 349 (KCl); 344 (DPBS); and 326 mOsm/kg (glucose). These data suggest that the osmolality values for NaCl and KCl were highest at the optimal concentration levels (approximately 350 to 380), but that similar osmolality obtained with DPBS or glucose (using: DPBS, 0.4 and 0.5×; glucose 90-100 mM) would lead to a less significant AAV increase.

For a more accurate measure, the osmolality of the various production media was also measured at the time of infection and supplementation, and during the course of the production cycle, as shown in FIG. 11D. Osmolality in the production media was measured at time of infection (0 hrs, n=5), after 24 hrs (n=3), and at harvest (48 hrs, n=4) after supplementation, five independent experiments. It was observed that the osmolality values for glucose and KCl were very similar and were slightly, but not significantly, lower than the ones obtained with NaCl, and that the osmolality increased over time (FIG. 11D). Altogether these data strongly suggest that the effect of NaCl and KCl, although correlated in part with an increase in osmolality of the production media, could not be entirely reproduced by other compounds such DPBS or glucose used at the same osmolality. This suggests that the effect of NaCl and KCl was specific to these two salts.

Lastly, the combination of KCl and glucose was tested at various concentration combinations as shown in FIG. 11E. For each condition, KCl and glucose concentrations are expressed as percentage of optimal concentration (40 mM for KCl and 80 mM for glucose; n=3). Similar to what was observed for the NaCl and KCl combination, no further increases were observed when glucose and KCl were combined. However, and also similar to what was obtained previously, an upward trend was noted with increasing concentrations of KCl in the combination, suggesting that KCl alone was a powerful additive to promote AAV titer increase.

### Salt Effect may be Specific to the HSV Platform

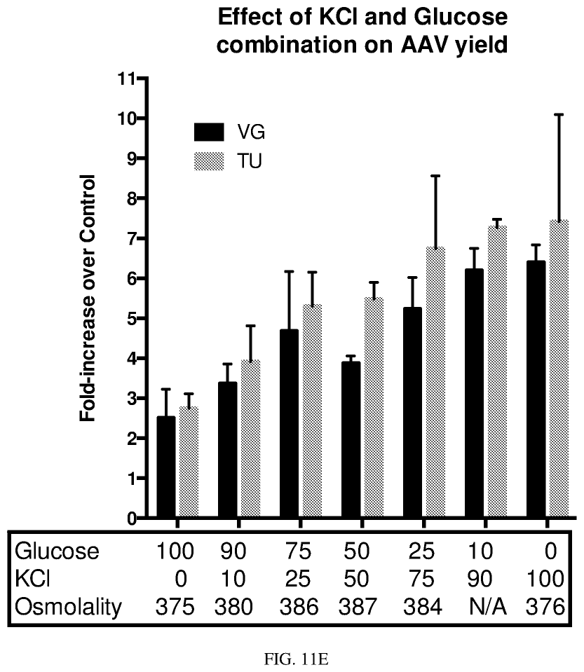

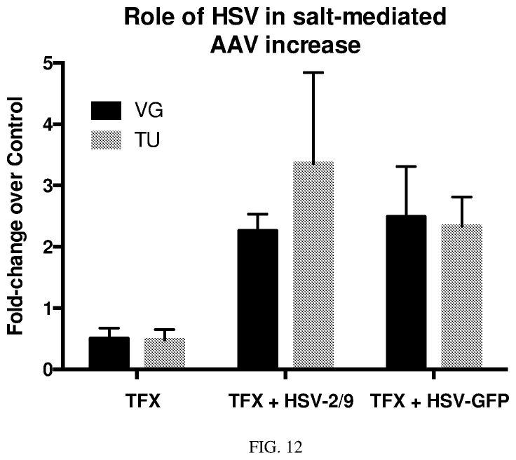

Lastly, experiments were conducted to test whether NaCl or KCl could improve AAV yield when produced by transfection. FIG. 12 shows the effect of salt on AAV yields when produced by transfection. AAV9-GFP was produced by transfection with two plasmids, pTRUF5 and pDG9-KanR (n=6), by transfection of the AAV genome (pTRUF5) in combination with the HSV helper (HSV-2/9; n=2), or by transfection of the helper plasmid pDG9-KanR and infection with the HSV-GOI (HSV-GFP; n=5). Experiments were performed in the presence or absence of salt (NaCl or KCl). NaCl or KCl were supplemented to the production medium at the time of transfection and AAV9-GFP yields were assessed. Ratios over the matching non-treated conditions are shown as an average of an independent experiments.

For a fair comparison, the same AAV-GFP genome was present in either plasmid pTRUF5 or HSV-GFP. AAV yields obtained by transfection were affected negatively by the salt supplementation in most cases. To rule out that the salt would impact the transfection efficiency, various time points of supplementation were tried, either before or several hours after transfection, but no further success was obtained. However, if transfection was combined with either of the two recombinant HSVs, an increase was observed. Altogether these data suggest that the salt effect may be entirely or partly mediated by the presence of HSV function in the producer cells. It remains possible that transfection could benefit from salt supplementation in conditions that have yet to be tested or identified.

### Conclusion

Altogether these results demonstrate that the observed increase in AAV titer results from the addition of NaCl or KCl in a specific, temporal, and non-synergistic manner. Upon using optimal concentrations of NaCl or KCl in the production media, the average volumetric yield of AAV in cell lysates was approximately 2 to 8×10¹⁴ vg/L based on small (50 mL) and large (3 L) scales, or approximately 2 to 8×10⁵ vg/cell. It was further observed that the increase in particle yield correlated with an increase in AAV particle potency, resulting in lower vg:tu ratios. This suggests that the particles produced were of improved quality.

Further, although the addition of salt affected the osmolality of the medium within a similar range for each of NaCl, KCl, DPBS, and glucose, a solid and significant correlation between the osmolality value and the increase in AAV yield was not observed. Additionally, the overall effect of glucose, although not negligible, was less than the one observed with any of NaCl, KCl, or DPBS. For this reason, it is concluded that the increase in AAV particle production is specifically linked to an effect triggered by the supplementation of KCl.

### Discussion

Increasing production yields will be paramount to ensure AAV success at commercial levels, both to sustain the demands for one-time high-dose or multi-dose products and to mitigate the cost per dose in favor of affordability. Though almost any production system can be expanded by increasing the number of bioreactors and/or the volume per batch, such changes are costly and ineffective. Typical rAAV specific yields often range around 1×10⁴ AAV vector genome (vg) per cell, far lower than the >1×10⁶ genome produced during the course of the wild-type AAV infection. Because it is difficult to reproduce the yields obtained with the wild-type virus in rAAV, an improvement that specifically increases the yield of vector produced per each producer cell, as shown herein, is highly valuable.

Unexpectedly, it was previously observed that the addition of sodium chloride to the production medium could increase AAV yield by more than one log when using an HSV infection system (Adamson-Small, et al., Hum Gene Ther Methods. 2017 Feb. 1; 28(1): 1-14). The results shown herein demonstrate that supplementation of potassium chloride, but no other salts tested, was able to achieve a similar or higher increase AAV yield than that which was observed upon supplementing sodium chloride. It is demonstrated herein that the observed increase was not due to an increase in cell number, cell viability, or size. This strongly supports the fact that the specific yield, or the production of AAV particles per cell, was significantly improved directly as a result of salt supplementation.

The observations herein demonstrate that infectious titers of the vector produced in the presence of salt consistently showed an increase, but particle numbers did not. The peak of AAV production when using the HSV system according to the methods described herein was observed to be between 24 and 48 hours. The effect of salt supplementation was observed to be temporal and transient, and likely acts within 8 to 16 hours post-infection. Because both HSV and AAV Rep proteins interfere with the cellular cell cycle, it is possible that this time frame is directly linked to a cell cycle phase induced by the HSV and/or the AAV replication in the producer cells.

The presently observed effects of salt supplementation on AAV yield could be mediated by NaCl and KCl ion channels and/or ion transporters. Base concentrations of most commercially available media (e.g., DMEM) likely contain approximately 5 mM KCl and 109.5 mM NaCl, respectively, based on the known formula for some standard culture media. Upon supplementing the KCl and/or NaCl at their optimal concentrations, as disclosed herein, the total concentrations of KCl and NaCl in the medium would increase to approximately 45 mM and 180-200 mM, respectively (including the addition of the HSV stocks). In mammalian cells, the intracellular concentration of KCl (˜139 mM) is about 10 times higher than that of NaCl (˜12 mM). Thus, according to the methods described herein, the extracellular media concentrations are lower for KCl and higher for NaCl than the intracellular environment. For KCl, the extracellular concentration was increased by approximately 8-fold, and for NaCl by approximately 2-fold.

The results described herein also detail the specific impact of increased osmolality during the production phase of rAAV. While similar studies addressing virus production are still very limited, osmotic shock, created by a sudden increase in the medium osmolality, is a commonly used strategy to increase production of monoclonal antibodies (mAbs). Most of these studies have shown that increased osmolality (typically above 300 mOsm) results in a sharp decrease in cell growth in any of Chinese hamster kidney cells (CHO), hybridomas, or HEK 293 cells. Since mAbs are typically produced from established mammalian cell lines that produce mAbs continuously, the overall production yield relies simultaneously on the total cell numbers, after a multi-day growth phase, and the specific yield, or protein production per cell. To overcome the negative effect of high osmolality on cell growth, a common mAb production strategy relies on a bi-phasic batch culture approach, with a growth phase under iso-osmotic conditions, followed by a production phase at high osmolality. In the methods of the present disclosure, growth and production are temporally separated: cells are amplified before the production phase, counted, and set at a defined density to ensure proper rHSV MOIs. Cell growth is limited thereafter. The effect of osmotic shock is known to be cell specific; the osmolality range varies depending on the cell line and the media used.

Despite these similarities, the methods of the present disclosure are unlike mAb production. Firstly, the increase in osmolality was not, at least solely, responsible for the increase in AAV production. It is well accepted that osmolality change is the direct cause for the increase that has been observed in mAb production. Secondly, the yield increase described herein was notably higher than the rate increase reported in mAb production. This is likely the result of critical differences between mAb and rAAV production: mAb production relies essentially on transcription and translation in cells that are selected for this performance. On the contrary, rAAV production involves genome rescue and replication, gene expression, and genome packaging, all of which are heavily controlled both by cellular and helper virus pathways. Even a modest increase in AAV replication could exponentially impact AAV gene expression and packaging.

The use of osmotic shock and/or salt supplementation as described herein surprisingly increased viral production. In fact, studies using HEK293 cells to produce adenovirus directly contradict the present findings, and have reported a decrease in yield in hyperosmotic media (see, e.g., Nadeau, I. et al. (1996), Biotechnol Bioeng 51: 613-623; Ferreira, T. B., et al. (2005), Biotechnol Lett 27: 1809-1813). In cases where an increase in adenovirus yield in hyperosmotic media was reported upon modulating the osmolality value between the growth and production phases (see, e.g., Shen et al. (2010), Biotechnol Prog 26: 200-07), the HEK293 cells were adapted to a higher osmolality (370 mOsm) by passaging prior to the production of adenovirus, and production needed to be performed under isotonic conditions (e.g., the extracellular environment contained equal or near equal amounts of salt as did the intracellular environment). This is in sharp contrast with the present results, which clearly demonstrate that the presence of salt is required during the production phase for rAAV.

### Example 3. The Effect of Supplementing KCl and NaCl Prior to Infection.

The goal of these experiments was to determine whether adding NaCl and KCl prior to initiating the production cycle (e.g., adding the HSV inoculum) would impact the effect of either or both salts on AAV production.

Expi293 cells were counted and seeded at approximately 2×10⁶ cells/mL in 25- or 50-mL culture volume. NaCl (60 mM) and KCl (40 mM) were added to the culture medium. Approximately 20 to 24 hours after salt addition, the treated or untreated Expi293 cells were counted and seeded at 1×10⁶ cells/mL in 25- or 50-mL culture volume, and were infected with the viral inoculum (HSV-GFP and HSV-AAV 2/9). In one condition, no additional NaCl or KCl supplement was added. In the second condition, NaCl and KCl were added to obtain a final concentration of 60 or 40 mM, respectively, depending on the volume of treated cells and HSV inoculum that was added. See Table 6 for details.

Cells were harvested after 2 days, lysed, and crude lysates were prepared after Benzonase digestion. The total amount of AAV9-GFP vector produced in each condition was determined by qPCR and transduction assay to determine the physical and infectious particle titers, respectively.

TABLE 6
Experimental design.

| Condition # | Cell treatment | NaCl | KCl |
|---|---|---|---|
| 1 | Untreated | − | − |
| 2 | Untreated | 60 mM | − |
| 3 | Untreated | − | 40 mM |
| 4 | NaCl pre-treated | − | − |
| 5 | NaCl pre-treated | + | − |
| 6 | KCl pre-treated | − | − |
| 7 | KCl pre-treated | − | + |

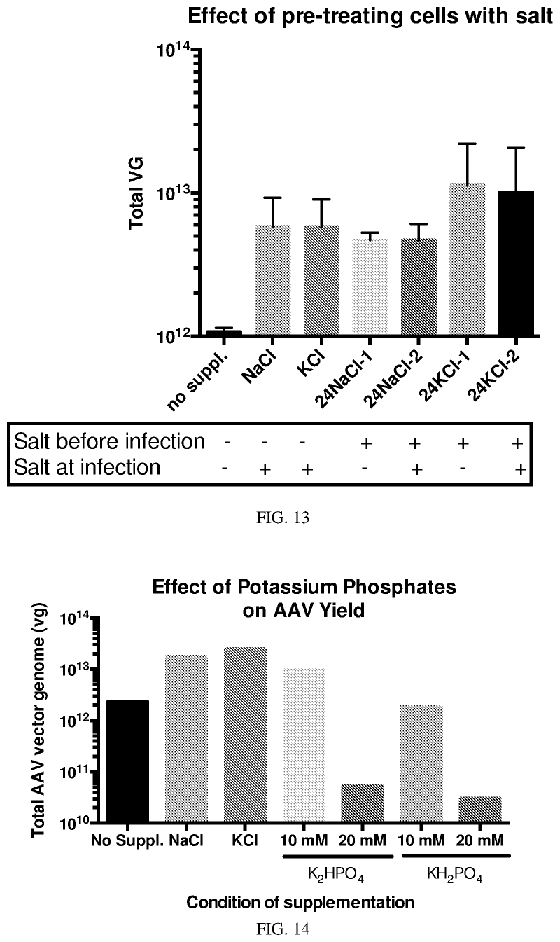

Exposing the cells to NaCl or KCl up to 24 hours prior to perform the infection did not impact the overall effect observed with NaCl or KCl (FIG. 13). The AAV production yield was either similar or increased in the two experiments that were conducted.

### Example 4. The Effect of Potassium and Sodium Alone, Respectively, Versus the Effect of Chloride Alone

The goal of these experiments was to determine whether the sodium and potassium components of NaCl and KCl were the active components, or whether the observed effect was mediated by the chloride component of each salt. Sodium and potassium phosphates were tested: Na₂HPO₄ (disodium phosphate) and potassium phosphate mono and dibasic (KH₂PO₄ and K₂HPO₄, respectively).

Pre-made solutions were purchased from Millipore-Sigma: 1 M solution, Potassium phosphate monobasic, Cat. No. P8709; 1 M solution, Potassium phosphate dibasic, Cat. No. P8584; and 0.5 M solution, sodium phosphate, dibasic, Cat. No. 95046. Expi293 cells were counted and seeded at 1×10⁶ cells/mL in 25 or 50 mL culture volume. Viral inoculum (HSV-GFP and HSV-AAV 2/9) was added directly to the cells, followed by the addition of the various supplements. Solutions containing the sodium and potassium phosphates were added to the cell culture media at various final concentrations (see FIGS. 14-18) ranging from 1 mM to 60 mM. Control conditions consisted of: cells infected without any supplement, cells infected in the presence of supplemented sodium chloride (NaCl, 60 mM final concentration), and cells infected in the presence of supplemented potassium chloride (KCl, 40 mM final concentration). Cells were harvested after 2 days, lysed, and crude lysates prepared after Benzonase digestion. The total amount of AAV9-GFP vector produced in each condition was determined by qPCR and transduction assay to determine the physical and infectious particle titers, respectively.

In the first experiment, concentrations from 10 to 60 mM were evaluated (see Table 7 for details of each condition).

TABLE 7
Conditions used in first experiment.

| Condition # | NaCl | KCl | K₂HPO₄ | KH₂PO₄ | Na₂HPO |
|---|---|---|---|---|---|
| 1 | — | — | — | — | — |
| 2 | 60 mM | — | — | — | — |
| 3 | — | 40 mM | — | — | — |
| 4 | — | — | 10 mM | — | — |
| 5 | — | — | 20 mM | — | — |
| 6 | — | — | 30 mM | — | — |
| 7 | — | — | 40 mM | — | — |
| 8 | — | — | 50 mM | — | — |
| 9 | — | — | — | 10 mM | — |
| 10 | — | — | — | 20 mM | — |
| 11 | — | — | — | 30 mM | — |
| 12 | — | — | — | 40 mM | — |
| 13 | — | — | — | 50 mM | — |
| 14 | — | — | — | — | 20 mM |
| 15 | — | — | — | — | 30 mM |
| 16 | — | — | — | — | 40 mM |
| 17 | — | — | — | — | 50 mM |
| 18 | — | — | — | — | 60 mM |

FIG. 14 shows the amount of AAV9-GFP produced after adding K₂HPO₄ (10 and 20 mM) or KH₂PO₄ (10 and 20 mM) to the production medium as compared to cells receiving either NaCl (60 mM final concentration) or KCl (40 mM final concentration) supplementation, or to cells not being supplemented. The data show that addition of K₂HPO₄ at 10 mM resulted in an increase in AAV production of about 4-fold when compared to non-supplemented cells. However, the increase was lower than the one observed for NaCl and KCl (approximately 8-fold). At concentrations above 10 mM, both K₂HPO₄ and KH₂PO₄ resulted in a dramatic reduction of more than 40-fold (20 mM), and in excess of 3,000-fold (>30 mM) in AAV production (data not shown).

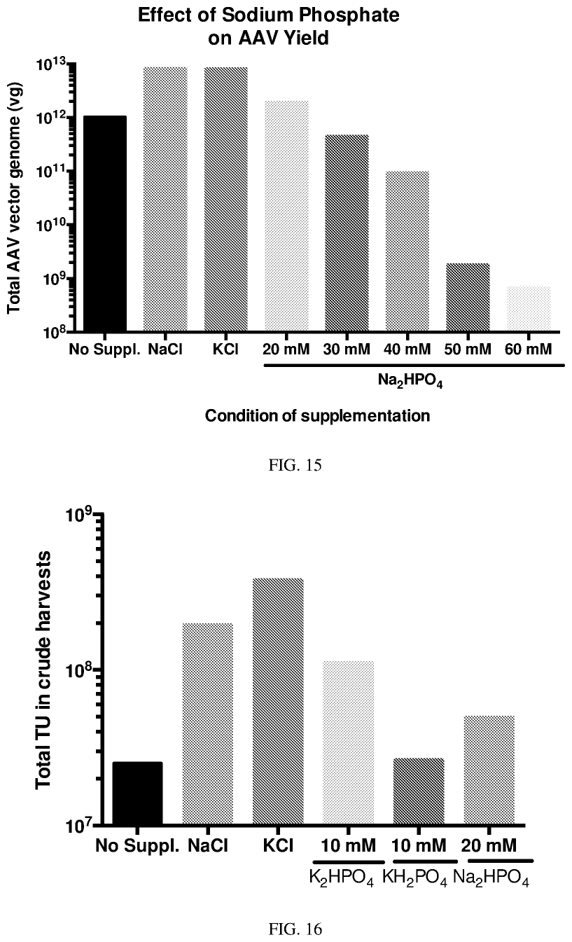

FIG. 15 shows the amount of AAV9-GFP produced after adding Na₂HPO₄ (20 to 60 mM) to the production medium as compared to cells receiving either NaCl (60 mM final concentration) or KCl (40 mM final concentration), or to cells not being supplemented. The data show that addition of Na₂HPO₄ at 20 mM resulted in a very modest (approximately 2-fold) increase in AAV production when compared to non-supplemented cells. However, the increase was significantly lower than the one observed for NaCl and KCl (approximately 8-fold). Concentrations higher than 30 mM resulted in a dramatic reduction in AAV production. These data were confirmed by the infectious unit titers (FIG. 16).

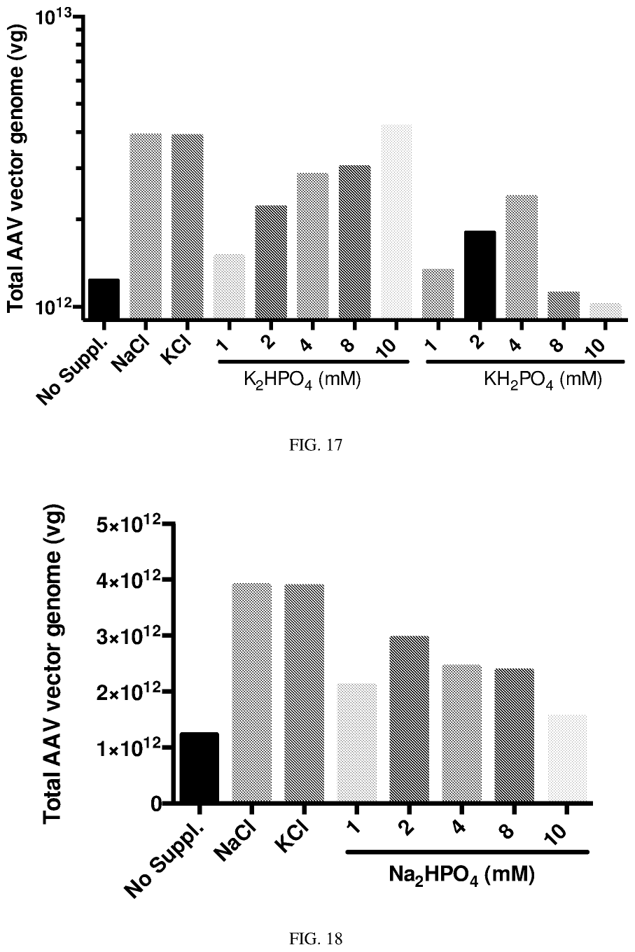

In a second set of experiments, lower concentrations (from 1 mM to 10 mM) of K₂HPO₄, KH₂PO₄, and Na₂HPO₄ were evaluated (see Table 8 for condition details; FIG. 17).

TABLE 8
Conditions used in second experiment.

| Condition # | NaCl | KCl | K₂HPO₄ | KH₂PO₄ | Na₂HPO |
|---|---|---|---|---|---|
| 1 | — | — | — | — | — |
| 2 | 60 mM | — | — | — | — |
| 3 | — | 40 mM | — | — | — |
| 4 | — | — | 1 mM | — | — |
| 5 | — | — | 2 mM | — | — |
| 6 | — | — | 4 mM | — | — |
| 7 | — | — | 8 mM | — | — |
| 8 | — | — | 10 mM | — | — |
| 9 | — | — | — | 1 mM | — |
| 10 | — | — | — | 2 mM | — |
| 11 | — | — | — | 4 mM | — |
| 12 | — | — | — | 8 mM | — |
| 13 | — | — | — | 10 mM | — |
| 14 | — | — | — | — | 1 mM |
| 15 | — | — | — | — | 2 mM |
| 16 | — | — | — | — | 4 mM |
| 17 | — | — | — | — | 8 mM |
| 18 | — | — | — | — | 10 mM |

FIG. 17 shows a linear increase of AAV production with K₂HPO₄ concentration ranging from 1 to 10 mM. The highest increase was shown at 10 mM (4-fold increase), similar to the one observed in the earlier experiment where the same concentration was tested. The maximal effect of KH₂PO₄ was observed at 4 mM, which showed a modest 2-fold increase. Concentrations above 8 mM showed no effect, or a sharp reduction in AAV production. The effect of Na₂HPO₄ was more modest, as before, with a concentration of 2 mM showing an increase less than 3-fold, and minimum to no effect observed at concentrations between 4 and 10 mM (FIG. 18).

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and, without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited. In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B”, the disclosure also contemplates the alternative embodiments “a composition consisting of A and B” and “a composition consisting essentially of A and B”.

What is claimed is:

1. A method of producing rAAV in mammalian cells, the method comprising:
(i) culturing mammalian cells in a medium;
(ii) adding one or more viral vectors encoding recombinant AAV (rAAV) genes and/or genes of interest into the medium; and
(iii) supplementing the medium with at least 5 mM potassium chloride.

2. The method of claim 1, wherein the mammalian cells are HEK293 cells, BHK cells, or HeLa cells.

3. The method of claim 1 or claim 2, wherein the recombinant AAV genes comprise one or more of AAV rep genes and/or AAV cap genes.

4. The method of any one of claims 1-3, wherein the medium is supplemented with between 5 and 100 mM potassium chloride. The method of any one of claims 1-3, wherein the medium is supplemented with between 20 and 100 mM potassium chloride.

6. The method of any one of claims 1-3, wherein the medium is supplemented with between 30 and 100 mM potassium chloride.

7. The method of any one of claims 1-3, wherein the medium is supplemented with between 30 and 50 mM potassium chloride.

8. The method of any one of claims 1-3, wherein the medium is supplemented with 40 mM potassium chloride.

9. The method of any one of claims 1-8, wherein the method further comprises:
(iv) supplementing the medium with 60 mM of sodium chloride.

10. The method of claim 9, wherein the adding of step (ii) and supplementing of steps (iii) and/or (iv) are performed at the same time.

11. The method of claim 9, wherein the supplementing of steps (iii) and/or (iv) is performed before the adding of step (ii).

12. The method of claim 11, wherein the supplementing of steps (iii) and/or (iv) is performed 2 hours before the adding of step (ii).

13. The method of claim 9, wherein the supplementing of steps (iii) and/or (iv) is performed after the adding of step (ii).

14. The method of claim 13, wherein the supplementing of steps (iii) and/or (iv) is performed between 1 minute and 8 hours after the adding of step (ii).

15. The method of any one of claims 9-14, wherein the supplemented potassium chloride and/or sodium chloride remains present for between 8 and 14 hours after the time at which the mammalian cells are contacted with the one or more viral vectors.

16. The method of any one of claims 1-15, further comprising diluting or changing the medium such that the concentration of potassium chloride is decreased.

17. The method of any one of claims 9-16, further comprising diluting or changing the medium such that the concentration of sodium chloride is decreased.

18. The method of claim 16 or claim 17, wherein diluting or changing the medium occurs between 0.5 and 8 hours after the time at which the mammalian cells are contacted with the one or more viral vectors.

19. The method of any one of claims 1-18, wherein the one or more viral vectors are one or more rHSV vectors.

20. The method of claim 19, wherein the one or more rHSV vectors comprise a first rHSV encoding a gene of interest flanked by AAV ITRs, and a second rHSV encoding AAV rep and cap genes.

21. The method of claim 20, wherein the AAV ITRs are AAV-2 ITRs.

22. The method of claim 20, wherein the AAV ITRs are from AAV-1, AAV-3, AAV-4, AAV-5, AAV-6, AAV-7, AAV-8, AAV-9, AAV-10, AAV-11, AAV-12, or any other AAV serotype.

23. The method of any one of claims 3-22, wherein the AAV cap gene is an AAV-1, AAV-3, AAV-4, AAV-5, AAV-6, AAV-7, AAV-8, AAV-9, AAV-10, AAV-11, AAV-12, or any other AAV serotype cap gene.

24. The method of any one of claims 3-23, wherein the AAV rep and AAV cap genes are operably linked to a promoter.

25. The method of any one of claims 1-24, wherein the mammalian cells are cultured in adherent format or suspension format.

26. The method of any one of claims 1-25, wherein the mammalian cells are cultured on micro-carriers that are in suspension.

27. A method of producing rAAV in mammalian cells, the method comprising contacting mammalian cells with one or more viral vectors encoding recombinant AAV (rAAV) genes and/or genes of interest in the presence of at least 6 mM potassium chloride.

28. The method of claim 27, wherein the potassium chloride is present in the medium in which the cells are contacted with the one or more viral vectors to introduce rAAV genes (the infection medium).

29. The method of claim 27 or claim 28, wherein the potassium chloride is present in the medium in which the cells produce rAAV particles (the producer medium).

30. The method of claim 28 or claim 29, wherein the producer medium is the same as the infection medium.

31. The method of any one of claims 27-30, wherein the rAAV genes comprise one or more of AAV rep genes and/or AAV cap genes.

32. The method of any one of claims 27-31, wherein the mammalian cells are contacted in the presence of between 6 and 100 mM potassium chloride.

33. The method of any one of claims 27-31, wherein the mammalian cells are contacted in the presence of between 20 and 100 mM potassium chloride.

34. The method of any one of claims 27-31, wherein the mammalian cells are contacted in the presence of between 30 and 100 mM potassium chloride.

35. The method of any one of claims 27-31, wherein the mammalian cells are contacted in the presence of between 30 and 50 mM potassium chloride.

36. The method of any one of claims 27-31, wherein the mammalian cells are contacted in the presence of 45 mM potassium chloride.

37. The method of any one of claims 27-36, wherein the method further comprises contacting the mammalian cells with 170 mM sodium chloride.

38. The method of claim 37, wherein the potassium chloride and/or sodium chloride is present before, at, or after the time at which the mammalian cells are contacted with the one or more viral vectors.

39. The method of claim 38, wherein the potassium chloride and/or sodium chloride is present 2 hours before the time at which the mammalian cells are contacted with the one or more viral vectors.

40. The method of claim 38, wherein the potassium chloride and/or sodium chloride is present at the time at which the mammalian cells are contacted with the one or more viral vectors.

41. The method of claim 38, wherein the potassium chloride and/or sodium chloride is present between 1 minute and 8 hours after the time at which the mammalian cells are contacted with the one or more viral vectors.

42. The method of any one of claims 37-41, wherein the potassium chloride and/or sodium chloride remains present for between 8 and 14 hours after the time at which the mammalian cells are contacted with the one or more viral vectors.

43. The method of any one of claims 28-42, further comprising diluting or changing the medium such that the concentration of potassium chloride is decreased.

44. The method of any one of claims 37-43, further comprising diluting or changing the medium such that the concentration of sodium chloride is decreased.

45. The method of claim 43 or 44, wherein the diluting or changing the medium occurs between 0.5 and 8 hours after the time at which the mammalian cells are contacted with the one or more viral vectors.

46. The method of any one of claims 27-45, wherein the one or more viral vectors are one or more rHSV vectors.

47. The method of claim 46, wherein the one or more rHSV vectors comprise a first rHSV encoding a gene of interest flanked by AAV ITRs, and a second rHSV encoding AAV rep and cap genes.

48. The method of claim 47, wherein the AAV ITRs are AAV-2 ITRs.

49. The method of claim 47, wherein the AAV ITRs are from AAV-1, AAV-3, AAV-4, AAV-5, AAV-6, AAV-7, AAV-8, AAV-9, AAV-10, AAV-11, AAV-12, or any other AAV serotype.

50. The method of any one of claims 31-49, wherein the AAV cap gene is an AAV-1, AAV-3, AAV-4, AAV-5, AAV-6, AAV-7, AAV-8, AAV-9, AAV-10, AAV-11, AAV-12, or any other AAV serotype cap gene.

51. The method of any one of claims 31-50, wherein the AAV rep and AAV cap genes are operably linked to a promoter.

52. The method of any one of claims 27-51, wherein the mammalian cells are cultured in adherent format or suspension format.

53. The method of any one of claims 27-52, wherein the mammalian cells are cultured on micro-carriers that are in suspension.

54. The method of any one of claims 27-53, wherein the mammalian cells are HEK293 cells, BHK cells, or HeLa cells.

55. A method of producing rAAV in mammalian cells, the method comprising:
(i) supplementing a medium with at least 5 mM potassium chloride; and
(ii) contacting the mammalian cells with one or more viral vectors encoding recombinant AAV (rAAV) genes and/or genes of interest in the supplemented medium.

56. The method of claim 55, wherein the medium is the medium in which the cells are contacted with the one or more viral vectors to introduce recombinant AAV genes (the infection medium).

57. The method of claim 55 or claim 56, wherein the medium is the medium in which the cells produce rAAV particles (the producer medium).

58. The method of claim 56 or claim 57, wherein the producer medium is the same as the infection medium.

59. The method of any one of claims 55-58, wherein the recombinant AAV genes comprise one or more of AAV rep genes and/or AAV cap genes.

60. The method of any one of claims 55-59, wherein the medium is supplemented with between 5 and 100 mM potassium chloride.

61. The method of any one of claims 55-59, wherein the medium is supplemented with between 20 and 100 mM potassium chloride.

62. The method of any one of claims 55-59, wherein the medium is supplemented with between 30 and 100 mM potassium chloride.

63. The method of any one of claims 55-59, wherein the medium is supplemented with between 30 and 50 mM potassium chloride.

64. The method of any one of claims 55-59, wherein the medium is supplemented with 40 mM potassium chloride.

65. The method of claims 55-64, wherein the method further comprises:
(iii) supplementing the medium with 60 mM sodium chloride.

66. The method of any one of claims 55-65, wherein the medium is supplemented before, at, or after the time at which the mammalian cells are contacted with the one or more viral vectors.

67. The method of claim 66, wherein the medium is supplemented 2 hours before the time at which the mammalian cells are contacted with the one or more viral vectors.

68. The method of claim 66, wherein the medium is supplemented at the time at which the mammalian cells are contacted with the one or more viral vectors.

69. The method of claim 66, wherein the medium is supplemented between 1 minute and 8 hours after the time at which the mammalian cells are contacted with the one or more viral vectors.

70. The method of any one of claims 65-69, wherein the supplemented potassium chloride and/or sodium chloride remains present for between 8 and 14 hours after the time at which the mammalian cells are contacted with the one or more viral vectors.

71. The method of any one of claims 55-70, further comprising diluting or changing the medium such that the concentration of potassium chloride is decreased.

72. The method of any one of claims 65-71, further comprising diluting or changing the medium such that the concentration of sodium chloride is decreased.

73. The method of claim 71 or claim 72, wherein the diluting or changing the medium occurs between 0.5 and 8 hours after the time at which the mammalian cells are contacted with the one or more viral vectors.

74. The method of any one of claims 55-73, wherein the one or more viral vectors are one or more rHSV vectors.

75. The method of claim 74, wherein the one or more rHSV vectors comprise a first rHSV encoding a gene of interest flanked by AAV ITRs, and a second rHSV encoding AAV rep and cap genes.

76. The method of claim 75, wherein the AAV ITRs are AAV-2 ITRs.

77. The method of claim 75, wherein the AAV ITRs are from AAV-1, AAV-3, AAV-4, AAV-5, AAV-6, AAV-7, AAV-8, AAV-9, AAV-10, AAV-11, AAV-12, or any other AAV serotype.

78. The method of any one of claims 59-77, wherein the AAV cap gene is an AAV-1, AAV-3, AAV-4, AAV-5, AAV-6, AAV-7, AAV-8, AAV-9, AAV-10, AAV-11, AAV-12, or any other AAV serotype cap gene.

79. The method of any one of claims 59-78, wherein the AAV rep and AAV cap genes are operably linked to a promoter.

80. The method of any one of claims 55-79, wherein the mammalian cells are cultured in adherent format or suspension format.

81. The method of any one of claims 55-80, wherein the mammalian cells are cultured on micro-carriers that are in suspension.

82. The method of any one of claims 55-81, wherein the mammalian cells are HEK293 cells, BHK cells, or HeLa cells.


SEQUENCE LISTING (2 sequences)

SEQ ID NO: 1 - DNA, 20 bp, Artificial Sequence
  Synthetic
ccaggtccacttcgcatatt

SEQ ID NO: 2 - DNA, 20 bp, Artificial Sequence
  Synthetic
gcgtgcaatccatcttgttc